(19) United States
(12) Patent Application Publication
(10) Pub. No.: US 2016/0281078 A1
(43) Pub. Date: Sep. 29, 2016

(54) METHODS FOR SEPARATING NUCLEIC ACIDS BY SIZE

(71) Applicant: Qiagen GmbH, Hilden (DE)

(72) Inventors: Roland Fabis, Hilden (DE); Nadine Krüger, Hilden (DE); Jan Petzel, Hilden (DE)

(21) Appl. No.: 15/160,801

(22) Filed: May 20, 2016

Related U.S. Application Data
(63) Continuation of application No. 14/347,444, filed on Mar. 26, 2014.

(30) Foreign Application Priority Data
Sep. 26, 2011 (EP) 11182811.7

(51) Int. Cl. C12N 15/10; B01D 15/42; B01D 15/36; C12Q 1/68; B01D 15/34

(52) U.S. Cl. CPC C12N 15/101; C12Q 1/6806; C12Q 1/6874; B01D 15/34; B01D 15/363; B01D 15/426; C12N 15/1013

(57) ABSTRACT

The present invention pertains to a method for isolating nucleic acids by size from a sample comprising nucleic acids of different sizes using an anion exchange matrix, wherein nucleic acids of a preselected size or a preselected size range are isolated by varying the pH value during elution and/or binding.

This application is a continuation of U.S. patent application Ser. No. 14/347,444, which is incorporated herein by reference, in its entirety.

### FIELD OF INVENTION

The present invention is related to the field of molecular biology, in particular the isolation of nucleic acids. The invention provides a method for separating nucleic acids by size from a sample comprising nucleic acids of different sizes. Therefore, the present invention provides means to isolate nucleic acids of a desired preselected size, respectively a preselected size range from a mixture of nucleic acids.

### BACKGROUND OF THE INVENTION

Methods for isolating nucleic acids are known in the prior art. Such methods involve separating nucleic acids of interest from other sample components, such as for example protein contaminations or potentially also other nucleic acids, also often referred to as non-target nucleic acids. If it is intended to isolate a specific nucleic acid of interest (also referred to as target nucleic acid) from other nucleic acids the separation process is usually based on differences in parameters of the target and the non-target nucleic acid such as for example their topology (for example super-coiled DNA from linear DNA), their size (length) or chemical differences (e.g. DNA from RNA) and the like.

For certain applications differences in size is an important criterion to distinguish target nucleic acids from non-target nucleic acids. Differences in size are e.g. important in order to isolate a specific nucleic acid fragment from a mixture of fragments or for isolating a specific type of nucleic acids or e.g. in order to isolate the shorter (smaller) extracellular nucleic acids separately from the longer intracellular nucleic acids. Several methods exist in order to isolate nucleic acids of a specific target size, respectively a specific target size range. A classic method for isolating nucleic acids of a target size involves the separation of the nucleic acids in a gel, and then isolating the nucleic acids of the target size from said gel. Respective methods are time consuming, as the portion of the gel containing the nucleic acids of interest must be cut out and then treated to degrade the gel or otherwise extract the nucleic acids of the target size from the gel slice. Furthermore, it is very difficult to isolate small nucleic acids such as e.g. extracellular nucleic acids and in particular small RNA using respective technologies.

As the interest in nucleic acids of a specific size has increased in the last years, several other methods were developed in order to isolate nucleic acids of a specific size. Examples include WO 05/012487 which describes a method for preparing nucleic acids of a preselected size using specific affinity columns and filtering technologies. Another method which allows to isolate nucleic acids of a specific size is described in WO 2007/140417, wherein nucleic acids are bound to a silica surface using a chaotropic salt solution. In a first step, the larger non-target nucleic acids are bound to the solid phase, the smaller nucleic acids remain in the supernatant. In the second step, smaller nucleic acids are bound to the solid phase and thereby are isolated from the supernatant. Another method for selectively isolating DNA and DNA fragments of various sizes from a sample is described in U.S. Pat. No. 6,534,262. A method for separating RNA by size is described in WO 2009/070465.

This prior art shows that there is an increasing interest and need in methods for isolating target nucleic acids of a desired target size, respectively a desired target size range.

Therefore, it is an object of the present invention to provide a method for isolating nucleic acids of a target size or a target size range from a sample comprising nucleic acids of different sizes.

### SUMMARY OF THE INVENTION

The present inventors have found that when using an anion exchange matrix, it is possible to isolate nucleic acids of a preselected target size or a preselected target size range from a mixture of nucleic acids of varying size (length) by varying the pH value during elution and/or binding. Thus, the present invention allows to isolate e.g. smaller nucleic acids separately from longer nucleic acids from a mixture comprising nucleic acids of different length (size). The cut-off value for the nucleic acid size can be controlled by adjusting the pH value during elution and/or binding.

Thus, the present invention provides a method for isolating nucleic acids by size from a sample comprising nucleic acids of different sizes using an anion exchange matrix, wherein nucleic acids of a preselected size or a preselected size range are isolated by adjusting the pH value during elution and/or binding. In preferred embodiments, the anion exchange matrix is provided by a solid phase which comprises anion exchange groups. Most preferred, magnetic particles are used as solid phase. In particular, the anion exchange matrix can be provided by magnetic particles comprising anion exchange groups on their surface.

According to a first embodiment of said method, nucleic acids of different sizes are bound to the anion exchange matrix. A size fractionation of the nucleic acids is achieved in this embodiment in the elution step. Here, smaller nucleic acids are selectively eluted from the anion exchange matrix by adjusting, respectively raising the pH value compared to the binding step. Because smaller nucleic acids have compared to longer nucleic acids less negatively charged phosphate groups which can interact with the anion exchange matrix, they bind less tightly to the anion exchange matrix than longer nucleic acids. Thus, they are eluted at a lower pH value compared to longer nucleic acids which remain due to their larger size bound to the anion exchange matrix at a pH value at which smaller nucleic acids are already eluted. Thus, the differential elution conditions used in this embodiment of the present invention allows to isolate smaller nucleic acids separately from longer nucleic acids. The cut-off value between smaller and longer nucleic acids depends on and thus can be determined by the chosen pH value as is also demonstrated in the examples. Thus, according to a first embodiment, a method for isolating nucleic acids by size from a sample comprising nucleic acids of different sizes is provided, wherein said method comprises the following steps:
a) binding nucleic acids of different sizes to an anion exchange matrix at a first pH wherein nucleic acids bind to the anion exchange matrix;
b) selectively eluting nucleic acids of a preselected size or a preselected size range from the anion exchange matrix using a second pH which is higher than the first pH and wherein the average length of the nucleic acids eluted from the anion exchange matrix is shorter than the average length of the nucleic acids which remain bound to the solid phase.

According to said embodiment, the present invention provides a method for separating nucleic acids by size (or length, these terms are used as synonyms herein) wherein selective elution conditions are used. Surprisingly, the present inventors have found that when using an anion exchange matrix for binding nucleic acids, nucleic acids of a preselected size, respectively a preselected size range can be eluted from the anion exchange matrix by adjusting respectively varying the elution pH value. Selective elution of the shorter nucleic acids is achieved by adjusting the concentration of positive charges on the surface of the anion exchange matrix so that predominantly longer nucleic acid molecules remain bound to the matrix during elution while smaller nucleic acids are eluted. The adjustment of the positive charges is controlled by the second pH value that is used during the elution step. At a certain elution pH, predominantly smaller (respectively shorter, these terms are used as synonyms herein) nucleic acid molecules will elute from the anion exchange matrix. When the elution pH is elevated, also longer nucleic acids can be eluted. Thus, the size of the nucleic acids that are eluted from the anion exchange matrix depends on the pH value that is used during elution. Because the chosen pH value influences the size of the nucleic acids that are eluted (smaller nucleic acids are compared to longer nucleic acids eluted at lower pH values) nucleic acids of a preselected size or a preselected size range can be specifically eluted by controlling or adjusting the pH value during elution. Thereby, nucleic acids can be separated according to their size. In particular, it was found that already small changes in the elution pH suffice to achieve this size fractionating effect during elution as is demonstrated in the examples.

According to a second embodiment, the size fractionation is achieved during the binding step. As discussed above, compared to longer nucleic acids smaller nucleic acids have less negatively charged phosphate groups which can interact with the positively charged groups on the anion exchange matrix. To achieve a size separation effect during the binding step, a pH value is chosen, wherein longer nucleic acids can bind to the anion exchange matrix, while smaller nucleic acids can not. Under the chosen selective binding conditions longer nucleic acids bind to the anion exchange matrix, while smaller nucleic acids do not bind and therefore, remain in the supernatant or flow-through. Thereby, smaller nucleic acids can be separated from longer nucleic acids. The cut-off value between smaller and longer nucleic acids depends again on and thus can be controlled by the chosen pH value as is also demonstrated in the examples. Thus, according to a second embodiment, a method for isolating nucleic acids by size from a sample comprising nucleic acids of different sizes is provided wherein said method comprises the following steps:
a) selectively binding nucleic acids of a preselected size or a preselected size range to an anion exchange matrix at a first pH, wherein the average length of the nucleic acids that bind to the anion exchange matrix under the chosen binding conditions is longer than the average length of nucleic acids which are not bound to the anion exchange matrix;
b) separating the bound nucleic acids from the remaining sample.

As described above, a separation of the nucleic acids according to their size can be achieved e.g. by varying respectively carefully adjusting the pH value during binding and/or elution. This principle that relies on the pH value to isolate nucleic acids of a specific size range has several advantages over prior art methods that are based e.g. on the use of chaotropic agents or alcohols. The separation of the nucleic acids according to their size can be performed according to the present invention e.g. without the use of flammable solvents, such as ethanol or other alcohols. The methods presented herein are also advantageous over polyethylene glycol based isolation methods because less washing steps are needed in order to purify the nucleic acids. In general, less washing is sufficient because for binding, an aqueous solution having a specific pH value can be used as compared to highly viscous polyethylene glycol. That fewer washing steps are needed is beneficial in order to save materials and time. Furthermore, no harmful chemicals such as e.g. chaotropic agents must be used in order to achieve a size separation of the nucleic acids, what is another advantage over prior art methods. Thus, according to one embodiment, the methods according to the present invention use binding conditions which do not involve the use of alcohols and/or chaotropic salts or other chaotropic agents.

The methods for isolating nucleic acids by size according to the present invention furthermore are highly precise and reproducible with respect to the size distribution of the isolated nucleic acids. Unexpectedly, this could be achieved despite of the low complexity of the methods. In particular, using one binding step and one selective elution step at one particular elution pH or using only one selective binding step at one particular binding pH, nucleic acids of the desired size can be obtained. Due to these simple and straight-forward binding and elution steps, the methods according to the present invention are optimally suitable for batch procedures, in particular using magnetic beads as anion exchange matrix. This also delimitates methods of the present invention from chromatographic methods which were previously used for size separation of nucleic acids. The methods of the present invention in particular do not need highly specific and expensive apparatuses, the generation and use of elution buffer gradients and the collection and screening of multitudes of elution fractions. A simple one container batch format using e.g. magnetic separation of the anion exchange magnetic particles is sufficient to provide nucleic acids of the desired size from complex samples when performing the size separation methods according to the present invention.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

The present invention provides a method for isolating nucleic acids by size from a sample comprising nucleic acids of different sizes using an anion exchange matrix, wherein nucleic acids of a preselected size or a preselected size range are isolated by varying the pH value during elution and/or binding. Nucleic acids have negatively charged phosphate groups which can interact with positively charged groups on the surface of an anion exchange matrix. Thereby, nucleic acids bind to the anion exchange matrix, in particular to magnetic particles bearing anion exchange groups.

The longer the nucleic acids, the more phosphate groups are available for interaction with the anion exchange surface and hence, the more efficient is the binding. Smaller nucleic acids have compared to longer nucleic acids fewer negatively charged phosphate groups that are available for interaction with the anion exchange matrix and thus, bind less tightly to the anion exchange matrix. Thus, to efficiently bind smaller nucleic acids to an anion exchange matrix, it is necessary to use strong binding conditions and thus a low pH value as otherwise, smaller nucleic acids will not bind efficiently to the anion exchange matrix. In contrast, longer nucleic acids can bind to the anion exchange matrix even if less strong binding conditions are used. This pH dependent difference in the binding behavior of smaller and longer nucleic acids is used in the present invention to separate and hence isolate nucleic acids according to their size by adjusting the pH value during elution and/or binding in order to achieve a size separation effect. Hence, the size fractionation of nucleic acids using an anion exchange matrix according to the present invention can be achieved by two main embodiments which will be explained in detail in the following.

According to a first embodiment, nucleic acids of different sizes are bound to the anion exchange matrix. Here, the size separation is achieved in the elution step, wherein size selective elution pH value is used under which nucleic acids of a preselected size or a preselected size range are eluted from the anion exchange matrix while longer nucleic acids remain bound to the anion exchange matrix. As discussed above, smaller nucleic acids have a lower amount of negatively charged phosphate groups and therefore are not bound as tightly to the anion exchange matrix compared to longer nucleic acids which comprise more negatively charged phosphate groups which can interact with the anion exchange matrix. Therefore, a pH value is chosen for elution under which (smaller) nucleic acids of a preselected size or size range are eluted while longer nucleic acids are not. The pH value that is used during elution according to the present invention depends on the desired cut-off value and hence on the desired size respectively size range of the nucleic acids that shall be eluted. As is shown by the examples, already small pH shifts of e.g. 0.1 or 0.2 pH units have an influence on the size of the nucleic acids that are eluted from an anion exchange matrix.

Thus, according to a first embodiment, a method for isolating nucleic acids by size from a sample comprising nucleic acids of different sizes is provided, wherein said method comprises the following steps:
a) binding nucleic acids of different sizes to an anion exchange matrix at a first pH wherein nucleic acids bind to the anion exchange matrix;
b) selectively eluting nucleic acids of a preselected size or a preselected size range from the anion exchange matrix using a second pH which is higher than the first pH and wherein the average length of the nucleic acids eluted from the anion exchange matrix is shorter than the average length of the nucleic acids which remain bound to the solid phase.

In binding step a), nucleic acids of different sizes are bound to the anion exchange matrix at a first pH which allows binding the nucleic acids to the anion exchange matrix. In binding step a), preferably binding conditions are used which allow to bind total nucleic acids (optionally of a specific type such as predominantly DNA or predominantly RNA) and therefore, nucleic acids of various sizes to the anion exchange matrix. In order to efficiently bind and thus capture longer as well as smaller nucleic acids, preferably strong binding conditions are used in step a). The first pH that is used during binding in order to efficiently capture nucleic acids of various sizes respectively the desired size range inter alia depends on the anion exchange matrix that is used for binding the nucleic acids. The binding conditions are preferably predominantly established via the pH value. Preferably, the pH value lies in a pH range from 3 to 10. Suitable ranges include but are not limited to 4 to 9, 4 to 8, 4 to 7, 4.5 to 6.5 and 5 to 6. Preferably, the pH value is below 7, more preferably below 6.5 and most preferably below 6. Lower pH values ensure that also smaller nucleic acids can be captured in the binding step a). The captured nucleic acids are then size fractionated in elution step b).

According to one embodiment, substantially all nucleic acid sizes that are represented in the sample are bound to the anion exchange matrix in step a). However, the pH value that is used in binding step a) can also be used to predominantly capture nucleic acids above a certain size. Hence, according to another embodiment, nucleic acids are predominantly bound to the anion exchange matrix in step a) which are longer than and thus which lie above a certain cut-off value. This allows to deplete e.g. very small nucleic acids (e.g. oligonucleotides or nucleic acids having a size below 50 nt or below 100 nt) in the binding step a) that are not of interest. Thus, according to one embodiment, the first pH value is chosen such so that nucleic acids are bound in step a) which have a size (chain length) greater than about 20 nt, greater than about 50 nt, greater than about 75 nt, greater than about 100 nt, greater than about 150 nt, greater than about 200 nt, greater than about 300 nt or in some embodiments even greater than about 500 nt. Further details and suitable conditions with respect to size selective binding conditions are also described in conjunction with the second embodiment according to the present invention. It is referred to the respective disclosure. The bound nucleic acids are then separated according to their size in elution step b).

In order to establish the binding conditions and in particular the first pH value, preferably a binding solution is added to the sample, thereby forming a binding mixture. The binding solution is according to one embodiment an aqueous solution which may also comprise salts or other organic compounds. Preferably the binding solution comprises at least one buffering compound and/or at least one acidifying compound in order to establish the desired binding conditions, in particular the first pH value in the binding mixture. Suitable buffering compounds and acidifying compounds can be selected from the group comprising acids, acidic buffering agents such as e.g. acetic acid, sodium acetate/acetic acid buffers, citric acid/citrate buffers, HCl, HClO₄, HClO₃, formic acid, boric acid, H₂SO₄, H₂SO₃, acidic phosphoric acid/phosphate buffer systems, biological buffers such as MES, MOPS, CHAPS, HEPES or other water-soluble inorganic or organic acids. The buffers also can contain chelators, such as NTA or EDTA, or organic compounds, such as alcohols, carbon hydrates, or urea. High salt ion concentrations, e.g. higher than 1 M, are not advantageous in order to maintain optimized binding conditions. An increase in the salt concentration results in a lower binding efficiency because the ionic bonds are weakened. This effect, however, can be compensated to a certain extent by a lower binding pH. However, preferably, the salt concentration in the binding solution or in the binding mixture is below 1M, below 0.75M, below 0.6M, below 0.5M, below 0.4M, below 0.35M, 0.3M or below 0.25M.

After binding step a), nucleic acids of different sizes are bound to the anion exchange matrix. The separation of the bound nucleic acids according to their size is achieved in elution step b). As discussed above, in elution step b), the size fractionation of the bound nucleic acids occurs by adjusting the second pH value so that longer nucleic acids can still bind to the anion exchange matrix (and thus are not eluted) while smaller nucleic acids can no longer bind and thus, are eluted from the anion exchange matrix. Smaller nucleic acids have less phosphate groups that can interact with the anion exchange matrix. Thus, smaller nucleic acids are eluted earlier than longer nucleic acids when raising the pH value. Thus, small nucleic acids can be eluted at a lower pH value than longer nucleic acids. The cut-off value and hence the size of the nucleic acids that can no longer bind to the anion exchange matrix and thus are selectively eluted depends on and thus can be controlled by the second pH value that is chosen for elution.

The second pH value that is used during elution is higher than the first pH. An elevation of the pH value results in a deprotonation of the anion exchange matrix. By reducing the number of positive charges on the anion exchange matrix, the interaction and thus binding to the negatively charged phosphate groups of the nucleic acids is weakened. This results in that the nucleic acids are released. As smaller nucleic acids have a lower amount of negatively charged phosphate groups than longer nucleic acids, smaller nucleic acids are released earlier and thus at a lower second pH value than longer nucleic acids. In order to efficiently elute longer nucleic acids, a higher pH value must be used which lies above the pH value that is sufficient to elute smaller nucleic acids. Hence, a size selective elution can be achieved by raising the pH value to a pH at which the desired cut-off value is achieved and hence, under which the nucleic acids of the desired target size or target size range are selectively eluted.

Depending on the size distribution of nucleic acids in a nucleic acid population, such as e.g. the bound or the eluted nucleic acids, the average nucleic acid length in a population of nucleic acid molecules may refer to that nucleic acid length at which half of the nucleic acid molecules in the population have a shorter length and the other half of the nucleic acid molecules have a greater length than the average nucleic acid length. However, the average nucleic acid length in a population of nucleic acid molecules may also refer to that nucleic acid length that occurs most frequently in the respective nucleic acid population. The latter option to determine the average nucleic acid length will usually be more appropriate and thus preferred for skewed nucleic acid populations wherein a certain nucleic acid size or a certain nucleic acid size range prevails. The length of the nucleic acids that are eluted and the length of the nucleic acids that remain bound to the anion exchange matrix can be controlled and thus varied by adjusting the elution conditions. Thus, it is possible to adjust the size, respectively the size range of the nucleic acids that are eluted or which remain bound thereby allowing to obtain nucleic acids of a preselected target size, respectively preselected target size range. The difference in the average length of the nucleic acids eluted from the anion exchange matrix with said second pH and the average length of the nucleic acids that remain bound to the anion exchange matrix at said second pH is according to one embodiment selected from at least about 50 nt, at least about 100 nt, at least about 150 nt, at least about 200 nt, at least about 250 nt, at least about 300 nt, at least about 350 nt, at least about 400 nt, at least about 450 nt, at least about 500 nt and at least about 750 nt. According to one embodiment, the average length of the nucleic acids that are at the second pH value eluted from the anion exchange matrix is in the range of from up to about 500 nt, about 10 nt to about 300 nt, preferably from about 20 nt to about 250 nt, from 20 nt to 200 nt, or from about 50 nt to about 150 nt.

According to one embodiment, nucleic acids having a length of up to about 500 nt, preferably up to about 300 nt, up to about 250 nt or up to about 200 nt are predominantly eluted from the anion exchange matrix in the first elution step at the second pH and/or nucleic acids having a length of about 500 bp or more, preferably about 700 nt or more, about 800 nt or more or about 1000 nt or more predominantly remain bound to the anion exchange matrix in said elution step b). According to one embodiment, the length of the nucleic acids that are eluted from the anion exchange matrix in step b) lies in a range of from about 10 nt to about 1000 nt, preferably from about 20 nt to about 750 nt, from 20 nt to 500 nt, from 50 nt to about 350 nt, from 50 nt to about 250 nt from about 50 nt to about 200 nt, from 20 nt to about 150 nt or from about 10 nt to about 100 nt. As described above, the length of the nucleic acids is indicated. Hence, if the nucleic acid is a double stranded molecule (e.g. DNA) the above indications with respect to the nt length of the double stranded molecule and hence the bp.

Preferably, an elution solution is used in step b) which is capable of providing the second pH during the elution step. During said elution step b) elution conditions are used which involve the use of a second pH which is higher than the first pH that was used during binding. By adjusting the pH value to achieve the desired cut-off value, a size selective elution is achieved wherein the average length of the nucleic acids that are eluted from the anion exchange matrix is shorter than the average length of the nucleic acids which remain bound to the solid phase. Thereby, the desired size fractionation effect is achieved. Preferably, a second pH is used during elution at which predominantly the longer nucleic acids of the desired/preselected size range remain bound to the anion exchange matrix while nucleic acids that are smaller than the desired cut-off value are predominantly eluted. The release and thus elution of the smaller nucleic acids from the anion exchange matrix is achieved according to this embodiment by using a second pH value which lies above the first pH value but at which the longer nucleic acids above a preselected cut-off value are predominantly not yet eluted.

The second pH value that is used during elution is higher than the first pH value and can be lower, equal to or above the pKa value of the anion exchange groups of the anion exchange matrix. The choice of the second pH value depends on the cut-off value that is desired for separating the nucleic acids according to their size. As is shown in the examples, the cut-off value can be varied by the choice of the pH value that is used during elution. This allows adjusting the elution conditions so that nucleic acids of a preselected size, respectively a preselected size range are predominantly eluted while nucleic acids having a larger (longer) size predominantly remain bound to the anion exchange matrix. The second pH value that is used during elution is higher than the first pH and preferably lies in a range that is selected from 4 to 14, 5 to 13, 5.5 to 12, 6 to 11, 6.2 to 10, 6.3 to 9.5, 6.4 to 9, 6.5 to 8.7, 6.5 to 8.5, 6 to 7 and 7 to 9. As discussed herein, the choice of the second pH value that is suitable for size selective elution also depends on the chosen anion exchange matrix and the binding properties of the anion exchange surface. Hence, the suitable pH range for the second pH value is dependent on the used anion exchange matrix and in particular depends on the nature of the type and properties of the anion exchange groups (e.g. their pKa value) as well as on the density of the anion exchange groups on the matrix surface, such as e.g. the density of the amine groups on the surface of a particle. Suitable pH values can be determined by the skilled person.

According to one specific embodiment, the second pH value that is used in step b) lies in the range of 6 to 7, more preferably in the range of 6 to 6.5. Said low pH range is particularly suitable to elute nucleic acids according to their size, in particular nucleic acids having predominantly a size or size range below 700 nt from an anion exchange matrix which carries amine groups such as spermine groups as anion exchange groups. Said pH range is in particular suitable if said anion exchange groups are present on the matrix surface with a low density and thus a density that allows to achieve the desired cut-off value at a pH range of 6 to 7. If a higher density of anion exchange groups on the surface is used, the second pH that is used for elution usually lies above 6.5, above 7.0 or above 8 as described above in order to elute nucleic acids according to their size, in particular nucleic acids having predominantly a size or size range below 700 nt. For selectively eluting longer nucleic acids, higher pH values are preferred.

According to one embodiment, the second pH is at least 0.2 pH unit higher than the first pH, preferably at least 0.3 units higher, more preferably at least 0.4 pH units, more preferably at least 0.5 pH units higher than the first pH. Preferably, the second pH is not more than 4 pH units, not more than 3.5 pH units, not more than 3 pH units, not more than 2.5 pH units, not more than 2 pH units, not more than 1.5 pH units, not more than 1 pH unit or not more than 0.75 pH units higher than the first pH value. The second pH may be below, at or above the pKa of a protonatable group of the anion exchange group. However, preferably, it is at least 1 unit below the pKa, more preferably at least 1.5 units below the pKa or at least 2 units below said pKa.

The elution solution may also comprise an organic or inorganic salt, such as for example an acetate, chloride, phosphate, borate, carbonate or sulphate salt. Suitable cations may be selected from alkaline, earth alkaline or transition metal ions such as for example Mn2+; Fe2+, Fe3+, Cu2+, or Co2+. Furthermore, the elution solution may comprise a buffering component. As buffering components for example organic cations such as TRIS, BIS-TRIS or ammonium salts can be used. Preferred buffering components include but are not limited to biological buffers such as TRIS, BIS-TRIS, MES, HEPES, MOPS, CHAPS, and tricin. In a preferred embodiment, the elution is performed under low salt conditions. According to one embodiment, the elution solution does not comprise more than 1 M salt, preferably not more than 0.75M salt, not more than 0.5 M salt, more preferably not more than 0.25 M salt.

The size fractionating elution according to the present invention allows to isolate longer nucleic acids separately from smaller nucleic acids. The cut-off value for separation can be controlled by the choice of the pH value as is shown by the examples. The nucleic acids that are longer than the cut-off value remain bound to the anion exchange matrix, while the nucleic acids that are smaller than the cut-off value are eluted and thus are present in the eluate. The eluted smaller nucleic acids are preferably separated from the anion exchange matrix to which the longer nucleic acids remain bound. The choice of the separation method depends on the used anion exchange matrix. Suitable methods to obtain the eluate include sedimentation, centrifugation and separation involving the use of a magnet, if a magnetic anion exchange matrix is used. Thereby, nucleic acids which are predominantly smaller than the preselected cut-off value can be separated from nucleic acids that are predominantly longer than the preselected cut-off value and can be obtained in form of an eluate.

Depending on the desired size, respectively the desired size range of the target nucleic acids, the smaller nucleic acids, the longer nucleic acids or both can be further processed. E.g. if desired, the longer nucleic acids which are still bound to the anion exchange matrix after elution step b) can afterwards be eluted if desired in a second elution step c). Any elution method can be used in step c). According to one embodiment, in said second elution step c), the pH value and/or the salt concentration is raised compared to the conditions that were used in elution step b). Thus, according to one embodiment, nucleic acids are eluted in a step c) by using a third pH value which lies above the second pH value. In a preferred embodiment, the third elution pH is at least 0.2 pH units, preferably at least 0.3 pH units, at least 0.4 pH units or at least 0.5 pH units higher than second pH value. For the third pH value higher alkaline pH values of at least 8.5, at least 9, at least 10, at least 11 or at least 12 can be used. The suitable third pH value also depends on the intended further application of the eluate; e.g. stronger alkaline pH values, such as a pH value of at least 12, can be used and are advantageous, e.g. if single-stranded DNA is needed or RNA contaminations have to be reduced.

If desired, a further size selective elution step can be performed in step c) e.g. in order to elute nucleic acids predominantly having a second preselected size, respectively size range which is longer than the size of the nucleic acids that were eluted in step b). Nucleic acids that are predominantly longer than said second preselected size or preselected size range remain bound to the anion exchange matrix. If desired, they may also be eluted in a further step.

However, the longer nucleic acids which remain bound to the anion exchange matrix after elution step b) may also be discarded if only the eluted small nucleic acids are of interest. E.g. the anion exchange matrix with the bound longer nucleic acids can be separated from the small nucleic acids and discarded. The anion exchange matrix with the bound nucleic acids may also be directly transferred into an amplification reaction in order to amplify nucleic acids. Suitable conditions which allow to perform an amplification reaction such as e.g. a PCR reaction while the nucleic acids are bound to the anion exchange matrix are for example described in WO 2011/037692. Further suitable uses and downstream applications of the nucleic acids isolated according to the teachings of the present invention are described below.

Optionally one or more washing steps are performed after the nucleic acids were bound to the anion exchange matrix in step a) and thus prior to step b). Washing helps to remove contaminations such as for example lipids, proteins, salt components and/or carbohydrates which may also have been bound to the anion exchange matrix. A suitable washing solution may comprise salts, water, organic molecules such as urea, or organic solvents such as ethanol or acetone. For the washing solution it is important to use washing conditions with respect to the pH value and the salt concentration so that the nucleic acids are not eluted but remain bound to the anion exchange matrix.

Furthermore, the method may comprise additional steps such as e.g. a pretreatment step prior to the binding step in order to degrade, e.g. lyse the sample. Details with respect to said optional lysis step are described below.

In preferred embodiments, elution step b) is performed with an elution solution such as an elution buffer having a constant pH value. In particular, no pH gradient is used for elution of the nucleic acids in step b). More preferably, elution step b) is performed with one particular elution buffer, i.e. the composition of the elution buffer is not varied during elution step b). Furthermore, preferably also the optional second elution step c) is performed with an elution solution such as an elution buffer having a constant pH value and in particular pH gradient is used for elution of the nucleic acids in step c). More preferably, elution step c) is performed with one particular elution solution, i.e. the composition of the elution solution is not varied during elution step c).

According to the second embodiment of the method according to the present invention, the separation of the nucleic acids is achieved via the binding conditions. Thus, according to said second embodiment, a method for isolating nucleic acids by size from a sample comprising nucleic acids of different sizes is provided wherein said method comprises the following steps:
a) selectively binding nucleic acids of a preselected size or a preselected size range to an anion exchange matrix at a first pH, wherein the average length of the nucleic acids that bind to the anion exchange matrix under the chosen binding conditions is longer than the average length of nucleic acids which are not bound to the anion exchange matrix;
b) separating the bound nucleic acids from the remaining sample.

According to said method, the binding conditions are adjusted such so that longer nucleic acids can bind to the anion exchange matrix, while smaller nucleic acids, which comprise less negatively charged phosphate groups that can interact with the anion exchange matrix are predominantly not bound and thus remain in the remaining sample, herein also referred to the supernatant or flow-through. Respective size selective binding conditions can be achieved by adjusting the pH value during binding. By separating the anion exchange matrix with the bound longer nucleic acids from the remaining sample, the longer nucleic acids are being separated from the smaller nucleic acids which remain in the sample.

In step a) the pH value is chosen such that predominantly longer nucleic acids that are above a certain cut-off value are being bound to the anion exchange matrix, while smaller nucleic acids which have a lower amount of negatively charged phosphate groups are predominantly not bound to the anion exchange matrix. The chosen first pH value influences the protonation status of the anion exchange matrix. At a low pH value, the anion exchange surface is heavily protonated and thus, longer as well as smaller nucleic acids can bind to the anion exchange matrix. However, if the first pH value is raised respectively is higher, the number of positive charges on the anion exchange matrix is reduced, what weakens the interaction and thus the binding to the negatively charged phosphate groups of the nucleic acids. As smaller nucleic acids have a lower amount of negatively charged phosphate groups than longer nucleic acids, smaller nucleic acids bind less efficiently to the anion exchange matrix than longer nucleic acids at a higher pH value. The cut-off value and hence the size of the nucleic acids that can bind to the anion exchange matrix can be determined and thus controlled by the chosen pH value as is shown in the examples. Therefore, nucleic acids of a preselected size, respectively a preselected size range can be isolated from a mixture of nucleic acids having different sizes using the method according to the second embodiment of the present invention.

The choice of a suitable first pH value to establish size selective binding conditions inter alia depends on the desired target size, respectively the desired target size range of the nucleic acids (and hence the chosen cut-off value) and the anion exchange matrix that is used for binding. The lower the pH value the stronger the binding of the nucleic acids to the solid phase. Therefore, if it is intended to bind predominantly longer nucleic acids, it is preferred to use a higher pH value, under which the smaller nucleic acids of the preselected size, respectively the preselected size range predominantly can not bind to the chosen anion exchange matrix.

Depending on the size distribution of nucleic acids in a nucleic acid population, such as e.g. the bound or the eluted nucleic acids, the average nucleic acid length in a population of nucleic acid molecules may refer to that nucleic acid length at which half of the nucleic acid molecules in the population have a shorter length and the other half of the nucleic acid molecules have a greater length than the average nucleic acid length. However, the average nucleic acid length in a population of nucleic acid molecules may also refer to that nucleic acid length that occurs most frequently in the respective nucleic acid population. The latter option to determine the average nucleic acid length will usually be more appropriate and thus preferred for skewed nucleic acid populations wherein a certain nucleic acid size or a certain nucleic acid size range prevails. The length of the nucleic acids that predominantly bind to the anion exchange matrix and the length of the nucleic acids that do not bind to the anion exchange matrix can be controlled and thus varied by adjusting the binding conditions. Thus, it is possible to adjust the size, respectively the size range of the bound nucleic acids thereby allowing to obtain nucleic acids of a preselected target size, respectively preselected target size range. The difference between the average length of the nucleic acids that bind to the anion exchange matrix at the first pH and the average length of the nucleic acids that do not bind to the anion exchange matrix is selected from at least about 50 nt, at least about 100 nt, at least about 150 nt, at least about 200 nt, at least about 250 nt, at least about 300 nt, at least about 350 nt, at least about 400 nt, at least about 450 nt, at least about 500 nt and at least about 750 nt. As shown in example 8, when a binding pH of 6.6 or 6.7 is used in conjunction with the respective type of anion exchange matrix, the DNA length cut-off between the flow-through and the eluate is situated between 200 and 250, so that the 200 bp DNA fragment can still be found in the eluate, while 250 bp-sized DNA can be detected in the flow-through. So the difference between the bound DNA and the DNA in the flow-through can be estimated as about 100.

According to one embodiment, the average length of the nucleic acids that bind to the anion exchange matrix in step a) is at least 100 bp, at least 150 bp, at least 200 bp, at least 250 bp, at least 300 nt, at least 400 nt, at least 500 nt, at least 600 nt, at least 650 nt, at least 700 nt, at least 750 nt, at least 800 nt, at least 850 nt, at least 900 nt, at least 950 nt or at least 1000 nt. As described above, the length of the nucleic acids is indicated. Hence, if the nucleic acid is a double stranded molecule (e.g. DNA) the above indications with respect to the nt length refers to bp.

According to one embodiment, a binding solution is added in order to establish the selective binding conditions, in particular the first pH value in the binding mixture. Preferably, the selective binding conditions are established via the pH value. Preferably, the first pH value lies in a pH range selected from 4 to 11, from 4 to 9, from 5 to 9 from 5 to 8, from 5 to 7 and from 5.7 to 6.7. A first pH value in the range between 5.7 and 6.6 is particularly preferred when using an anion exchange matrix comprising spermine groups as is shown in the examples. As discussed above, the type and the density of the anion exchange groups that are present on the surface of the matrix influence the binding of the nucleic acids to said surface and hence the choice of the first pH value that is suitable for achieving the size fractioning effect.

As is shown in the examples, already small changes in the binding pH can have an impact on the cut-off value and hence the size, respectively the size range of the nucleic acids that can be bound to the anion exchange matrix.

It is preferred to use an aqueous binding solution. Said binding solution may optionally comprise further components such as salts or organic compounds. Preferably, the binding solution comprises at least one buffering compound and/or at least one acidifying compound in order to establish the desired binding conditions, in particular the desired first pH value. Suitable buffering compounds and acidifying compounds can be selected from the group comprising acids, acidic buffering agents such as e.g. acetic acid, sodium acetate/acetic acid buffers, citric acid/citrate buffers, HCl, HClO₄, HClO₃, formic acid, boric acid, H₂SO₄, H₂SO₃, acidic phosphoric acid/phosphate buffer systems, biological buffers such as MES, HEPES, CHAPS, MOPS, TRIS or BSI-TRIS or water-soluble inorganic or organic acids. High salt ion concentrations, e.g. higher than 1M, are not advantageous in order to maintain optimized binding conditions. An increase in the salt concentration results in a lower binding efficiency because the ionic bonds are weakened. This effect, however, can be compensated to a certain extent by a lower binding pH. The examples shown herein use buffer conditions with salt concentrations lower than 250 mM, but this invention is not limited to these salt quantities. E.g. the salt concentration in the binding solution or in the binding mixture can be below 1M, below 0.75M, below 0.6M, below 0.5M, below 0.4M, below 0.35M, 0.3M or below 0.25M. However, higher salt concentrations require a lower pH for binding, due to their influence in weakening the ionic interaction between anion exchange matrix and the DNA phosphate backbone.

After the size selective binding step, nucleic acids of the preselected target size, respectively the preselected target size range are predominantly bound to the anion exchange matrix while nucleic acids that are smaller than the preselected target size range predominantly remain in the remaining sample. Depending on the desired target size of the nucleic acids, either the smaller nucleic acids that remain in the supernatant or flow through can be further processed and/or the longer nucleic acids that were bound to the anion exchange matrix. According to one embodiment, the smaller nucleic acids that were not bound to the anion exchange matrix under the chosen binding conditions and thus remained in the supernatant or flow through are isolated therefrom using any suitable nucleic acid isolation method. Methods for isolating small nucleic acids are known in the prior art and are e.g. described in WO 07/100934, WO 2005/054466 and WO 11/086195. Commercially available kits for isolating small nucleic acids from various samples include but are not limited to Wizard® SV 96 PCR Clean-Up System (Promega), Agencourt AMPure XPMirvana miRNA Isolation Kit (Ambion). However, said method can also be used to selectively deplete smaller nucleic acids below a certain cut-off value from a mixture of nucleic acids by not binding them to the anion exchange matrix.

The bound longer nucleic acids may also be isolated, e.g. eluted from the anion exchange matrix using any suitable elution method. Non-limiting examples are described below. However, it is also within the scope of the present invention to discard the anion exchange matrix with the bound longer nucleic acids if only the small nucleic acids are of interest or if the method is used to selectively deplete or remove longer nucleic acids of a preselected size range.

Selective binding of the nucleic acids having the preselected size, respectively the preselected size range above the desired cut-off value is achieved during binding by adjusting the concentration of positive charges on the surface of the anion exchange matrix so that the nucleic acid molecules of the preselected size or preselected size range predominantly bind to the anion exchange matrix while smaller nucleic acids predominantly do not bind. The adjustment of the positive charges and hence the cut-off value of nucleic acids that can still bind to the anion exchange matrix is controlled by the first pH value that is used during the size selective binding step. In particular, size selective binding can be performed by adding a binding solution which is capable of establishing the first pH during the binding step. Details are described above.

If an elution of the bound nucleic acids is desired, any elution principle known in the prior art can be used. Preferably, elution is achieved by raising the pH value. According to one embodiment a second pH value is used during elution, which is higher than the first pH value and wherein under said conditions at least a portion of the nucleic acids that were bound to the anion exchange matrix are eluted. An elevation of the pH value results in a deprotonation of the anion exchange matrix. By reducing the number of positive charges on the anion exchange matrix, the binding of the nucleic acids is weakened. This results in that the bound nucleic acids are released. In preferred embodiments, said elution is performed with an elution solution such as elution buffer having a constant pH value. In particular, no pH gradient is used for elution of the nucleic acids. More preferably, elution is performed with one particular elution solution, i.e. the composition of the elution solution is not varied during elution.

Raising the salt concentration also allows to establish conditions under which at least a portion or even all of the bound nucleic acids is eluted. Higher salt concentrations disturb the interaction of the nucleic acids with the anion exchange matrix, thereby promoting elution. Furthermore, elution can also be established by combining a suitable second pH value with a suitable salt concentration in order to elute the bound nucleic acids. Elution can also be assisted by heating and shaking.

Preferably, elution conditions are used which involve the use of a second pH which is higher than the first pH that was used during binding. The release and thus elution of the nucleic acids from the anion exchange matrix is achieved according to this embodiment by raising the pH value. The second pH value that is used during elution is higher than the first pH value and can be lower, equal to or higher than the pKa value of the anion exchange groups of the anion exchange matrix. Preferably, the second pH value that is used during elution is higher than the first pH and preferably lies in a range that is selected from 4 to 14, 4 to 13, 5 to 12, 5.5 to 11, 6 to 10, 6.1 to 9.5, 6.2 to 9 and 6.2 to 8.7. Preferably, the second pH value that is used in step b) lies in the range of 6 to 9.5, more preferably 6.1 to 9 and most preferred in the range of 6.2 to 8.7. According to one embodiment, the second pH value lies at least 0.2 pH units, at least 0.3 pH units, at least 0.4 pH units, at least 0.5 pH units, at least 1 pH unit, at least 1.5 pH units or at least 2 pH units higher than the first pH value.

The elution solution may also comprise an organic or inorganic salt, such as for example acetate, chloride, phosphate, borate, carbonate or sulphate. Suitable cations may be selected from alkaline, earth alkaline or transition metal ions such as for example Mn2+; Fe2+, Fe 3+, Cu2+, or Co2+. Furthermore, the elution solution may comprise a buffering component. As buffering components for example organic cations such as TRIS, BIS-TRIS or ammonium salts can be used. Preferred buffering components include but are not limited to biological buffers such as TRIS, BIS-TRIS, MES, HEPES, MOPS and tricin.

Furthermore, to achieve an efficient elution of the nucleic acids that were bound in the size selective binding step can be achieved by using at least one polyanionic compound such as e.g. a carboxylate or polycarboxylate as are described for example in WO 2011/037692. Therefore, the elution solution may comprise one or more compounds selected from the group comprising oxalate, citrate, acrylate, polyacrylate, polymethacrylate, dextran sulfate, carboxymethyl cellulose, polybenzoic acid, polystyrene sulfate, polymaleic acid, mellitic acid and polymellitic acid. Further suitable examples of polyanionic compounds that can be used in order to assist the elution are described in WO 2011/037692, herein incorporated by reference.

The elution step can be performed such, that substantially all nucleic acids are eluted. However, if a further size fractionation of the bound nucleic acids is desired, a size selective elution step can be performed as is described above. Here, the elution conditions are chosen such that at the second pH value predominantly smaller nucleic acids are eluted while longer nucleic acids predominantly remain bound to the solid phase. The cut-off value can be controlled by the choice of the pH value as is described above in conjunction with the first embodiment according to the present invention. Longer nucleic acids may also be released from the anion exchange matrix in an optional second elution step if desired.

Furthermore, after binding optionally one or more washing steps can be performed. Suitable washing steps are described above, it is referred to the above disclosure. Furthermore, the method may comprise additional steps such as e.g. a pretreatment step prior to binding in order to degrade, e.g. lyse the sample. Details with respect to said optional lysis step are described below.

As discussed above, both embodiments of the method according to the present invention allow to efficiently separate nucleic acids according to their size, wherein the pH value that is used during binding and/or elution determines the cut-off value. Further characteristics and embodiments of the method are described below. This disclosure equally applies to the embodiment wherein the size fractionation is achieved during elution as well as to the embodiment wherein the size fractionation is achieved during binding if not indicated otherwise.

The anion exchange matrix that is used in the method according to the present invention preferably comprises a solid phase which carries anion exchange groups on the surface. However, soluble polymers such as are described in EP 1 345 952 or US 2008/0299557 may also be used. The nucleic acids bind unspecifically, i.e. independent of their sequence to the anion exchange groups. However, the use of a solid anion exchange matrix is preferred. The anion exchange groups preferably comprise at least one protonatable group. Any solid phase suitable for anion exchange chromatography may be used, including but not limited to silica and polysilicic acid materials, borosilicates, silicates, inorganic glasses, organic polymers such as poly(meth)acrylates, polyurethanes, nylon, polystyrene, polymers made of epoxides, amines, alkenes, polyamides, polycarbonates, agarose, polysaccharides such as cellulose, metal oxides such as silicon dioxide, ferrous oxide, aluminum oxide, magnesium oxide, titanium oxide and zirconium oxide, metals such as gold, titan, silicon or platinum, sephadex, sepharose, polyacrylamide, divinyl benzene polymers, styrene divinyl benzene polymers, dextrans, and derivatives thereof; hydrogels such as agarose, dextran, glass or silica surfaces. Preferred formats of the solid phase include but are not limited to particles, beads, membranes, filters, plates, and capillary tubes. In some embodiments, the anion exchange groups can be linked to the surfaces of processing vessels such as micro-tubes, tubes, pipette tips, monolithic columns, wells of micro-plates, or capillaries, and using these surfaces nucleic acids can be isolated also on a micro scale. The solid phase preferably is made of or contains a mineral or a polymeric material such as silica, glass, quartz, polyethylene, polypropylene, polybutylene, polyvinylidene fluoride, polyacrylonitrile, polyvinylchloride, polyacrylate, methacrylate or methyl methacrylate. Preferably, at least the surface carrying the anion exchange groups is composed of one of these materials or a mixture thereof, preferably silica materials and/or glass.

Furthermore, the solid phase may comprise a magnetic material. The use of magnetic particles is particularly preferred. Examples include but are not limited to magnetic (e.g. paramagnetic, superparamagnetic, ferromagnetic or ferrimagnetic) particles, including but not limited to polystyrene, agarose, polyacrylamide, dextran, and/or silica and polysilicic acid materials having a magnetic material incorporated therein or associated therewith. The magnetic material may be ferrimagnetic, ferromagnetic, paramagnetic or superparamagnetic and preferably is superparamagnetic or ferromagnetic. Preferably the magnetic material is completely encapsulated e.g. by the silica, polysilicic acid, glass or polymeric material. In certain preferred embodiments, the nucleic acid binding matrix is a polysilicic acid particle or a polymeric particle, preferably a magnetic polysilicic acid particle or a polymeric particle which carries anion exchange groups.

Furthermore, the anion exchange matrix according to the present invention can be an object or device, comprising an anion-exchange functionalized surface. E.g. it is possible to modify the surfaces of the consumables such as, for example, reaction vessels, reaction filters, filter columns, spin filter minicolumns, membranes, frits, glass fibre fabric, dishes, tubes, (pipette) tips or wells of multiwall plates for the binding, coated microtiter plates, coated tubes or other containers, monolithic columns or packed columns, consisting of anion-exchange-modified particles. In an especially preferred embodiment, magnetic particles are used.

When using magnetic particles, preferably, the separation is preferably achieved by the aid of a magnet. According to one embodiment, the nucleic acid binding matrix carrying the nucleic acid is magnetically attracted to the bottom or to a wall of the reaction vessel containing the reaction mixture and then the remaining reaction mixture is removed from the reaction vessel, for example by suction or decanting, or the magnetic particles are removed from the reaction vessel by plunging a magnet (which preferably is comprised in a cover or coating) into the reaction vessel and removing the magnetic particles from the remaining sample.

The anion exchange groups preferably are attached to the surface of a solid phase material. Coupling can be achieved by covalent attachment, non-covalent attachment or electrostatic attachment. Preferably, covalent coupling is used. Hence, the solid phase may be functionalized for attachment of the anion exchange groups, for example with functionalities such as Si—O—Si, Si—OH, alcohol, diol or polyol, carboxylate, amine, phosphate or phosphonate groups. The anion exchange groups may be attached to the solid phase, for example, by using epoxides, (activated) carboxylic acids, silanes, acid anhydrides, acid amides, acid chlorides, formyl groups, tresyl groups, tosyl groups or pentafluorophenyl groups, sulfonyl chloride or Maleinimide groups. The functional groups may be attached directly to the solid phase or via (linear or branched) spacer groups, e.g. hydrocarbons such as —(CH₂)ₙ— groups, carbohydrates, polyethylene glycols and polypropylene glycols. Alternatively, also a polymer composed of monomers comprising the anion exchange group such as e.g. an amino functional group can be used as anion exchange material. In certain embodiments, the solid phase material has a polysilicic acid surface and the anion exchange groups are coupled to the polysilicic acid surface using a silane group. Preferably, magnetic particles are used which comprise a coating with silica, polysilicic acid, glass or polymeric material to which the anion exchange groups are covalently attached, optionally via a linker group.

Anion exchange materials that can be used in the context of the present invention include, but are not limited to, materials modified with anion exchange groups. Examples of such anion exchange groups are groups comprising cations or polycations such as e.g. ammonium ions or phosphonium ions. Preferred examples of anion exchange groups include monoamines, diamines, polyamines, and nitrogen-containing aromatic or aliphatic heterocyclic groups. Preferably, the anion exchange group comprises at least one primary, secondary or tertiary amino group, more preferably at least one primary or secondary amino group. In preferred embodiments, the anion exchange group comprises a group selected from the group consisting of primary, secondary and tertiary amines of the formula

R₃N, R₂NH, RNH₂ and/or X—(CH₂)ₙ—Y

wherein
X is R₂N, RNH or NH₂,
Y is R₂N, RNH or NH₂,
R is independently of each other a linear, branched or cyclic alkyl, alkenyl, alkynyl or aryl substituent which may comprise one or more heteroatoms, preferably selected from O, N, S and P, and
n is an integer in the range of from 0 to 20, preferably 0 to 18.

Hence, the anion exchange groups may have a protonatable group and optionally may have more than one protonatable group which may be the same or different from each other. A protonatable group preferably is a chemical group which is neutral or uncharged at a high pH value and is protonated at a low pH value, thereby having a positive charge. In particular, the protonatable group is positively charged at the first pH in the methods according to the present invention at which binding of the nucleic acid to the solid phase occurs. Preferably, the pKa value of the (protonated) protonatable group is in the range of from about 7 to about 13, more preferably from 8 to about 13, more preferably from about 8.5 to about 12 or from about 9 to about 11.

Hence, examples of suitable anion exchange groups are in particular amino groups such as primary, secondary and tertiary amino groups as well as cyclic amines, aromatic amines and heterocyclic amines, preferably tertiary amino groups. Primary and secondary amino groups are especially preferred. The amino groups preferably bear alkyl, alkenyl, alkynyl and/or aromatic substituents, including cyclic substituents and substituents which together with the nitrogen atom form a heterocyclic or heteroaromatic ring. The substituents preferably comprise 1 to 20 carbon atoms, more preferably 1 to 12, 1 to 8, 1 to 6, 1 to 5, 1 to 4, 1 to 3 or 1 or 2 carbon atoms. They may be linear or branched and may comprise heteroatoms such as oxygen, nitrogen, sulfur, silicon and halogen (e.g. fluorine, chlorine, bromine) atoms. Preferably, the substituents comprise not more than 4, more preferably not more than 3, not more than 2 or not more than 1 heteroatom.

Particular examples of amine functions are primary amines such as aminomethyl (AM), aminoethyl (AE), aminoalkyl, alkylaminoalkyl, dialkylaminoalkyl such as diethylaminoethyl (DEAE), ethylene diamine, diethylene triamine, triethylene tetraamine, tetraethylene pentaamine, pentaethylene hexaamine, trimethylamino (TMA), triethylaminoethyl (TEAE), linear or branched polyethylene imine (PEI), carboxylated or hydroxyalkylated polyethylene imine, jeffamine, spermine, spermidine, 3-(propylamino)propylamine, polyamidoamine (PAMAM) dendrimers, Tris, Bis-Tris, polyallylamine, polyvinylamine, N-morpholinoethyl, polylysine, and tetraazacycloalkanes as well as cyclic amines and aromatic amines that are protonatable and can form an ammonium ion. Preferably, the anion exchange groups are selected from spermine and spermidine.

In one embodiment the anion exchange group preferably carries 1 to 10 amino groups. More preferably the anion exchange groups carries 2 to 8, and particularly the anion exchange group carries 2 to 6 amino groups. In preferred embodiments, the anion exchange group comprises a group selected from the group consisting of primary, secondary and tertiary mono- and poly-amines of the formula

R₁R₂R₃N,
R₁R₂N(CH₂)ₙNR₃R₄,
R₁R₂N(CH₂)ₙNR₃(CH₂)ₘNR₄R₅,
R₁R₂N(CH₂)ₙNR₃(CH₂)ₘNR₄(CH₂)ₒNR₅R₆
R₁R₂N(CH₂)ₙNR₃(CH₂)ₘNR₄(CH₂)ₒNR₅(CH₂)ₚNR₆R₇
R₁R₂N(CH₂)ₙNR₃(CH₂)ₘNR₄(CH₂)ₒNR₅(CH₂)ₚNR₆(CH₂)_{q}
NR₇R₈
R₁R₂N(CH₂)ₙNR₃(CH₂)ₘNR₄(CH₂)ₒNR₅(CH₂)ₚNR₆(CH₂)_{q}
NR₇(CH₂)ᵣNR₈R₉
R₁R₂N(CH₂)ₙNR₃(CH₂)ₘNR₄(CH₂)ₒNR₅(CH₂)ₚNR₆(CH₂)_{q}
NR₇(CH₂)ᵣNR₈(CH₂)ₛNR₉R₁0

wherein
m, n, o, p, q, r and s independently from one each other can be 2 to 8, R₁, R₂, R₃, R₄, R₅, R₆, R₇, R₈, R₉ and R₁₀ can be identical or different and are chosen from the group H, alkyl (branched or unbranched, saturated or unsaturated, preferably comprising 1 to 10 C atoms) and aryl.

In some preferred embodiments, the anion exchange group comprises N-propyl-1,3-propandiamine or pentaethylene hexamine. More preferred, the anion exchange groups are selected from spermine and spermidine.

The present invention also includes anion exchange matrices consisting of polyoxyalkylene amines with one, two or three amino groups. Those nucleic acid binding groups are available with the name “jeffamine” polyoxyalkylene amine. Such jeffamines comprise primary amino groups which are bound to the terminus of the polyether backbone. Said polyether backbone may be based on propylene oxide, ethylene oxide or mixtures thereof. Also other backbone segments are possible.

According to the present invention any mixtures of herein described anion exchange groups can be used to provide anion exchange matrix and to bind the nucleic acids. However, it is also within the scope of the present invention to predominantly or exclusively use one type of anion exchange matrix described herein.

In case the anion exchange group is attached to a solid phase via a covalent bond (directly or via a linker group), the anion exchange groups defined herein comprise an attachment point for said covalent bond, e.g. by deletion of an hydrogen atom or of an R-group of the anion exchange group.

The anion exchange group is preferably attached to the solid phase via a linker group. Thus, the anion exchange group in particular comprises a protonatable group attached to a linker group. The linker group preferably is a linear, branched or cyclic alkylene alkenylene or alkynylene group which preferably comprises 1 to 20 carbon atoms, more preferably 1 to 12, 1 to 8, 1 to 6, 1 to 5, 1 to 4, 1 to 3 or 1 or 2 carbon atoms. It may further comprise heteroatoms such as oxygen, nitrogen, sulfur, silicon and halogen (e.g. fluorine, chlorine, bromine) atoms, preferably not more than 4, more preferably not more than 3, not more than 2 or not more than 1 heteroatom. In preferred embodiments, the linker group is an alkylene group, in particular a propylene group. In some embodiments of the present invention, the anion exchange groups described herein may be bound to a solid surface material by covalent linkage or by electrostatic, polar or hydrophobic interaction. Preferentially the anion exchange groups are bound to the carrier surface such that each bound exchange group exposes 1 to 10 (in the case of for example N-propyl-1,3-propandiamin), preferred 2 to 8 and particularly preferred 2 to 6 amino groups. In preferred embodiments, the anion exchange groups are bound to a solid surface material—such as magnetic particles as described herein—via covalent bonds, optionally using a linker group between the anion exchange group and the solid surface as described above.

In certain embodiments, the solid phase further comprises inert ligands. Preferably, the inert ligands are not significantly involved in nucleic acid binding and/or do not strongly bind to nucleic acids. In particular, the inert ligands are neutral or uncharged and preferably are hydrophilic. The inert ligands may be attached to the nucleic acid binding matrix as described herein for the nucleic acid binding ligands. Preferably, the inert ligands are organic moieties, in particular alkyl, alkenyl, alkynyl or aromatic moieties which may be linear, branched or cyclic and which preferably comprise at least one heteroatom such as oxygen, nitrogen, sulfur, silicon and halogen (e.g. fluorine, chlorine, bromine) atoms. The inert ligand preferably comprises 1 to 20 carbon atoms, more preferably 1 to 12, 1 to 8, 1 to 6, 1 to 5, 1 to 4, 1 to 3 or 1 or 2 carbon atoms, and up to 4, more preferably up to 3, up to 2, or up to 1 heteroatom. Particularly preferred are inert ligands comprising at least one hydroxyl group, in particular at least 2 hydroxyl groups, such as ligands comprising a 2,3-dihydroxypropyl group. A specific example of the inert ligands is a (2,3-dihydroxypropyl)oxypropyl group.

The inert ligands can be used to reduce the amount of nucleic acid binding anion exchange groups which will attach to the solid phase during coupling of the anion exchange groups. Hence, the density of the anion exchange groups on the surface of the solid phase can be controlled and adjusted to the subsequent application of the solid phase. The lower the density of the anion exchange groups on the surface of the solid phase, the lower is the binding capacity to small nucleic acids as less positively charged groups are available for interaction. A reduced density of positively charge groups result in weaker ionic interaction to phosphate backbones and, thus weaker binding. In particular, the solid phase may comprise anion exchange groups and inert ligands in a ratio in the range of from about 1:1 to about 1:10, preferably from about 1:2 to about 1:5, more preferably about 1:3. In some embodiment, the solid phase does not comprise such inert ligands. According to this embodiment, the solid phase only comprises the anion exchange groups and does not comprise any other ligands.

Furthermore, the solid phase may comprise functional groups which assist and thus promote the elution at the elution pH value. Preferably, said functional groups are negatively charged at the elution pH value. E.g. the pKa values of said groups can lie in a range of 0 to 7, preferably 1 to 5. Suitable examples include ion exchangers, in particular cation exchangers, preferably acidic groups such as carboxyl groups. Further suitable groups include betains, sulfonate, phosphonate and phosphate groups. As described above, the solid phase may comprise carboxyl groups in order to allow the attachment of the anion exchange groups. When attaching the anion exchange groups, the concentration of the anion exchange groups can be chosen such that at least a portion of the carboxyl groups is not bound to anion exchange groups. These carboxyl groups do not disturb the binding of the nucleic acids if the binding pH value is sufficiently low. However, at higher pH values they become negatively charged, thereby promoting the release of the nucleic acids. Further suitable designs of the anion exchange matrix which promote the elution of the nucleic acids e.g. by not saturating the surface of the solid phase with anion exchange groups are described in WO 2010/072821.

Examples of suitable nucleic acid binding solid phases, anion exchange groups, protonatable groups and inert ligands are described in WO 2010/072834, WO 2010/072821 and DE 10 2008 063 003 and the respective disclosure is incorporated herein by reference.

According to a particularly preferred embodiment, the anion exchange matrix comprises a carboxylated surface comprising amine groups as anion exchange groups, preferably amine groups comprising 2 to 6 amino groups, most preferred comprising spermine groups. Preferably, said anion exchange matrix is provided in form of particles. When using a respective anion exchange matrix in the first embodiment of the present invention wherein a size selective elution is performed, the first (binding) pH is preferably below 6, most preferred about 5.8. At this pH value, nucleic acids of different sizes are efficiently bound to the anion exchange matrix. Preferably, the second (elution) pH is above 6, preferably in a range of about 6.2 to 6.4. Here, the cut-off value is at a chain length below 1000 nt, in particular at about 700 nt, i.e. nucleic acids having an average length below 700 nt are selectively eluted. When using a respective anion exchange matrix in the second embodiment of the present invention wherein a size selective binding step is performed, the first (binding) pH is preferably in a range from 6.5 to 7, preferably 6.6 to 6.8. Preferably, the second (elution) pH is above 8, preferably 8.5. Here, the cut-off value is at a chain length of about 200 nt, i.e. nucleic acids having an average length above 200 nt are selectively bound.

According to a further particularly preferred embodiment, the anion exchange matrix comprises a silica surface comprising aminoalkylsilane groups as anion exchange groups and dihydroxypropyloxy-propylsilanes. Preferably, said anion exchange matrix is provided in form of particles. When using a respective anion exchange matrix in the first embodiment of the present invention wherein a size selective elution is performed, the first (binding) pH is preferably below 6.5, most preferred about 6. At this pH value, nucleic acids of different sizes are efficiently bound to the anion exchange matrix. Preferably, the second (elution) pH is above 6, preferably in a range of about 6.5 to 6.7. Here, the cut-off value is at a chain length of about 500 nt, i.e. nucleic acids having an average length below 500 nt are selectively eluted. When using a respective anion exchange matrix in the second embodiment of the present invention wherein a size selective binding step is performed, the first (binding) pH is preferably in a range from 6.5 to 7, preferably 6.6 to 6.8. Preferably, the second (elution) pH is above 8, preferably in a range of 8.5 to 9. Here, the cut-off value is at a chain length of about 500 nt, i.e. nucleic acids having an average length above 500 nt are selectively bound.

The term “sample” is used herein in a broad sense and is intended to include a variety of sources that contain nucleic acids. The methods according to the first and second aspect can be used to isolate nucleic acids from a variety of samples. E.g. the sample may be a biological sample but the term also includes other, e.g. artificial samples which comprise nucleic acids such as e.g. amplification products or samples comprising nucleic acids for sequencing reactions. Exemplary samples include, but are not limited to, body fluids in general, whole blood; serum; plasma; red blood cells; white blood cells; buffy coat; swabs, including but not limited to buccal swabs, throat swabs, vaginal swabs, urethral swabs, cervical swabs, throat swabs, rectal swabs, lesion swabs, abcess swabs, nasopharyngeal swabs, and the like; urine; sputum; saliva; semen; lymphatic fluid; liquor; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, cell cultures, as well as environmental samples such as soil or water, food, lysates, extracts, or materials obtained from any cells and microorganisms and viruses that may be present on or in a sample and the like. Materials obtained from clinical or forensic settings that contain nucleic acids are also within the intended meaning of the term sample. Furthermore, the skilled artisan will appreciate that modified samples such as e.g. stabilized samples, lysates, extracts, or materials or portions thereof obtained from any of the above exemplary samples are also within the scope of the term sample. Preferably, the sample is a biological sample derived from a human, animal, plant, bacteria or fungi. Preferably, the sample is selected from the group consisting of cells, tissue, bacteria, virus and body fluids such as for example blood, blood products such as buffy coat, plasma and serum, urine, liquor, sputum, stool, CSF and sperm, epithelial swabs, biopsies, bone marrow samples and tissue samples, preferably organ tissue samples such as lung and liver and stabilized or fixed forms of the foregoing. According to one embodiment, the sample is selected from whole blood and blood products such as buffy coat, serum or plasma. Respective samples derived from blood are usually provided in a stabilized form, e.g. stabilized at least by the addition of an anticoagulant such as e.g. EDTA or a citrate salt.

Depending on the sample type, the sample can be optionally pretreated prior to step a) in the methods according to the present invention e.g. in order to make the nucleic acids available for binding. E.g. in said pretreatment step, nucleic acids can be released from cells or can be freed from other components such as e.g. proteins. Herein, we refer to a respective pretreatment step to degrade the sample generally as lysis step irrespective of whether nucleic acids are released from cells or whether the lysis is performed in order to release the nucleic acids e.g. from proteins or other substances. Several methods are known in the prior art that allow to achieve an efficient lysis of different sample types. Suitable lysis methods include but are not limited to mechanical, chemical, physical or enzymatic actions on the sample. Examples of respective lysis steps include but are not limited to grinding the sample in a bead mill, the application of ultrasound, heating, the addition of detergents and/or the addition of protein degrading compounds such as e.g. protein degrading enzymes, e.g. hydrolases or proteases or salts, e.g. chaotropic salts. According to one embodiment, a protein degrading compound is used during lysis. According to a preferred embodiment, the protein-degrading compound is a proteolytic enzyme. A proteolytic enzyme refers to an enzyme that catalyzes the cleavage of peptide bounds, for example in proteins, polypeptides, oligopeptides and peptides. Exemplary proteolytic enzymes include but are not limited to proteinases and proteases in particular subtilisins, subtilases, alkaline serine proteases and the like. Subtilases are a family of serine proteases, i.e. enzymes with a serine residue in the active side. Subtilisins are bacterial serine protease that has broad substrate specificities. Subtilisins are relatively resistant to denaturation by chaotropic agents, such as urea and guanidine hydrochloride and anionic detergents such as sodium dodecyl sulfate (SDS). Exemplary subtilisins include but are not limited to proteinase K, proteinase R, proteinase T, subtilisin, subtilisin A, QIAGEN Protease and the like. Discussions of subtilases, subtilisins, proteinase K and other proteases may be found, among other places in Genov et al., Int. J. Peptide Protein Res. 45: 391-400, 1995. Preferably, the proteolytic enzyme is proteinase K. Preferably, the proteolytic enzyme is used under heating and/or agitation. Suitable lysis conditions are known in the prior art and thus, need no detailed description here. The lysis conditions used must be chosen such that they do not interfere with the subsequent pH dependent binding step. Thus, it should be in particular ensured that the salt concentration is not too high. Therefore, preferably, lysis occurs under conditions wherein the salt concentration of the substances that are added for lysis is preferably below 1M, more preferred below 0.75M, more preferred below 0.5M and most preferred below 0.3M.

The term “nucleic acid” or “nucleic acids” as used herein, in particular refers to a polymer comprising ribonucleosides and/or deoxyribonucleosides that are covalently bonded, typically by phosphodiester linkages between subunits, but in some cases by phosphorothioates, methylphosphonates, and the like. Nucleic acids include, but are not limited to all types of DNA and/or RNA, e.g. gDNA; circular DNA; plasmid DNA; circulating DNA; PNA; LNA, cyclohexene nucleic acids; RNA/DNA hybrids; hnRNA; mRNA; noncoding RNA (ncRNA), including but not limited to rRNA, tRNA, miRNA (micro RNA), sRNA (small interfering RNA), snoRNA (small nucleolar RNA), snRNA (small nuclear RNA), pwi-interacting RNA (piRNA), repeat associated RNA (rasiRNA), as RNA and stRNA (small temporal RNA); fragmented nucleic acid; nucleic acid obtained from subcellular organelles such as mitochondria or chloroplasts; and nucleic acid obtained from microorganisms, parasites, or DNA or RNA viruses that may be present in a biological sample, e.g. bacteria, viral or fungi nucleic acids; synthetic nucleic acids, extracellular nucleic acids, amplification products, digestion products, PCR fragments, cDNA, oligonucleotides. The term “extracellular nucleic acids” or “extracellular nucleic acid” as used herein, in particular refers to nucleic acids that are not contained in cells. Respective extracellular nucleic acids are also often referred to as cell-free nucleic acids. These terms are used as synonyms herein. The term “extracellular nucleic acids” refers e.g. to extracellular RNA as well as to extracellular DNA. Examples of typical extracellular nucleic acids that are found in the cell-free fraction (respectively portion) of biological samples such as body fluids such as e.g. blood plasma include but are not limited to mammalian extracellular nucleic acids such as e.g. extracellular tumor-associated or tumor-derived DNA and/or RNA, other extracellular disease-related DNA and/or RNA, epigenetically modified DNA, fetal DNA and/or RNA, small interfering RNA such as e.g. miRNA and sRNA, and non-mammalian extracellular nucleic acids such as e.g. viral nucleic acids, pathogen nucleic acids released into the extracellular nucleic acid population e.g. from prokaryotes (e.g. bacteria), viruses or fungi. According to one embodiment, the extracellular nucleic acid is obtained from a body fluid as cell-containing biological sample such as e.g. blood, plasma, serum, saliva, urine, liquor, sputum, lachrymal fluid, sweat, amniotic or lymphatic fluid. Herein, we refer to extracellular nucleic acids that are obtained from circulating body fluids as circulating extracellular or circulating cell-free (ccf) nucleic acids. According to one embodiment, the term extracellular nucleic acid in particular refers to mammalian extracellular nucleic acids, preferably disease-associated or disease-derived extracellular nucleic acids such as tumor-associated or tumor-derived extracellular nucleic acids, extracellular nucleic acids released due to inflammations or injuries, in particular traumata, extracellular nucleic acids related to and/or released due to other diseases, or extracellular nucleic acids derived from a fetus. The term “extracellular nucleic acids” or “extracellular nucleic acid” as described herein also refers to extracellular nucleic acids obtained from other samples, in particular biological samples other than body fluids. Synthetic nucleic acid sequences may or may not include nucleotide analogs.

As becomes apparent from the described examples of samples that can be processed according to the method of the present invention, a sample may comprise more than one type of nucleic acid. Depending on the intended use, it may be desirous to isolate all types of nucleic acids from a sample (e.g. DNA and RNA) or predominantly certain types or predominantly a certain type of nucleic acid (e.g. predominantly RNA but not DNA or vice versa, or DNA and RNA are supposed to be obtained separately from a sample). All these variants are within the scope of the present invention. Suitable methods for isolating either DNA or RNA or both types of nucleic acids in parallel or together as total nucleic acid are known in the prior art.

The nucleic acids of the desired size, respectively the desired target size range (also referred to as target nucleic acid) that can be isolated using the methods according to the present invention can be directly analysed and/or further processed using suitable assay and/or analytical methods. The isolated target nucleic acids can be identified, modified, contacted with at least one enzyme, amplified, reverse transcribed, cloned, sequenced, contacted with a probe and/or be detected. Respective methods are well-known in the prior art and are also commonly applied in the medical, diagnostic and/or prognostic field in order to analyse or identify isolated nucleic acids or a specific nucleic acid comprised in the isolated nucleic acids. Thus, after the nucleic acids of the target size, respectively the target size range were isolated, they can be analysed to identify the presence, absence or severity of a disease state including but not being limited to a multitude of neoplastic diseases, in particular premalignancies and malignancies such as different forms of cancers. E.g. the isolated nucleic acids of the target size or the target size range can be analysed in order to detect diagnostic and/or prognostic markers (e.g., fetal- or tumor-derived extracellular nucleic acids) in many fields of application, including but not limited to non-invasive prenatal genetic testing respectively screening, disease screening, oncology, cancer screening, early stage cancer screening, cancer therapy monitoring, genetic testing (genotyping), infectious disease testing, testing for pathogens, injury diagnostics, trauma diagnostics, transplantation medicine or many other diseases and, hence, are of diagnostic and/or prognostic relevance. According to one embodiment, the isolated nucleic acids of the target size are analyzed to identify and/or characterize a disease infection or a fetal characteristic. Thus, the methods described herein may further comprise a step of nucleic acid analysis and/or processing of the isolated nucleic acids of the target size or target size range. The analysis/further processing of the nucleic acids can be performed using any nucleic acid analysis/processing method including, but not limited to identification technologies, amplification technologies, polymerase chain reaction (PCR), isothermal amplification, reverse transcription polymerase chain reaction (RT-PCR), quantitative real time polymerase chain reaction (Q-PCR), digital PCR, gel electrophoresis, capillary electrophoresis, mass spectrometry, fluorescence detection, ultraviolet spectrometry, hybridization assays, DNA or RNA sequencing, restriction analysis, reverse transcription, NASBA, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. Respective technologies are well-known to the skilled person and thus, do not need further description here.

Furthermore, the methods of the present invention can be used in the course of ligation-based methods for selective nucleic acid detection and amplification, for example in “next generation” sequencing technologies. Such high-throughput sequencing techniques parallelize the general sequencing process, resulting in the generation of thousands or millions of sequences at once. Commonly used “next generation” sequencing approaches include polony sequencing, 454 pyro-sequencing, IIlumina sequencing, SOLiD sequencing, ion semiconductor sequencing, DNA nanoball sequencing or sequencing by hybridization. During the sample preparation process DNA fragments of a defined size are generated, for example by DNA shearing (e.g., derived from using a nebulizer, sonicator or hydroshear). Said DNA fragments are then separated from a mixture of fragments so that DNA libraries of individual fragments with a defined length can be produced. In next generation sequencing methods, target DNA fragments to be sequenced can be ligated with adapter fragments that may in turn function as binding sites for sequencing primers or for attachment to a solid phase. Following a ligation reaction with adaptors precise fractionation of DNA fragments with a defined length is extremely important to select only those fragments that carry the ligated adaptor(s). The methods according to the present invention can be advantageously used in order to isolate DNA fragments of a defined size from a mixture of fragments. Hence, they can be used to separate ligated target DNA fragments inter alia from surplus non-ligated adapter-fragments and from serially fused adapter-adapter ligation products. The ability to produce a library comprising sheared DNA fragments, characterized by a narrow size distribution enables the investigator to construct a map of the original pre-sheared DNA molecule.

More specifically, the methods of the present invention can be used for sample preparation within “next generation” sequencing approaches targeting a genome, an epigenome or a transcriptome, for example for the purpose of de novo sequencing, targeted resequencing, whole genome resequencing, chromatin immunoprecipitation sequencing (ChIP), methylation analysis, small RNA analysis, gene expression profiling or whole transcriptome analysis.

Unless defined otherwise herein, all technical and scientific terms used herein have the same meaning as commonly understood by one of ordinary skill in the art to which this invention belongs. This invention is not limited by the exemplary methods and materials disclosed herein, and any methods and materials similar or equivalent to those described herein can be used in the practice or testing of embodiments of this invention. Numeric ranges are inclusive of the numbers defining the range. Numeric ranges include a tolerance range of +/−10%, +/−5%, preferably +/−3% and most preferred +/−1%. According to one embodiment, the numeric ranges include no tolerance range.

The term “solution” as used herein, e.g. as binding solution or lysis solution in particular refers to a liquid composition, preferably an aqueous composition. It may be a homogenous mixture of only one phase but it is also within the scope of the present invention that a solution that is used according to the present invention comprises solid components such as e.g. precipitates.

The term “predominantly” as used herein, e.g. when describing that nucleic acids are or remain predominantly bound to the anion exchange matrix or are predominantly eluted from the anion exchange matrix, in particular refers to a situation wherein at least 70%, at least 80%, at least 90%, at least 95% or at least 98% of the respective nucleic acids behave accordingly, e.g. are or remain bound to the anion exchange matrix or are eluted from the anion exchange matrix.

The invention is now illustrated by the following non-limiting examples:

### EXAMPLES

### Example 1

### Synthesis of Anion-Exchange-Modified Magnetic Carboxylate Beads

500 mg magnetic beads (Carboxyl-Adembeads, Ademtech, #02111, or Dynal MyOne Carboxy beads, #65011, Seradyn Sera-Mag Magnetic Carboxylate modified beads, or Seradyn Sera-Mag SpeedBeads) were resuspended in 10 ml 50 mM MES buffer, pH6.1. Then, 11.5 ml of a 50 mg/ml solution of N-hydroxysulfosuccinimide (NHS) were added and the solution was mixed with a mini-shaker. Then, 10 ml of a 52 μmol/l solution of 1-ethyl-3-(3-dimethylaminopropyl)carbodiimide (EDC) were added, followed by another mixing step. The reaction mixture was incubated for 30 min on a rotating end-over end shaker. The magnetic beads were separated with a magnet and the supernatant was removed. The beads were resuspended in 50 ml 50 mM MES buffer, pH6.1 and distributed to 5 aliquots of 10 ml each. The beads were separated with a magnet and the supernatants were removed. In each aliquot the beads were resuspended in 1 ml 50 mM MES buffer, pH 6.1. Each aliquot was supplemented with 2 ml of the corresponding amine solution, e.g. spermine, at a concentration of 500 mg/ml in 50 mM MES buffer, pH 8.5 or 9.0. The ingredients of the aliquots were carefully mixed and treated with ultrasound for 10 min. Then the aliquots were incubated on a rotating end-over end shaker for 1 hr. The beads were washed with 10 ml 50 mM MES buffer, pH6.1, followed by separation with the magnet and removal of the supernatants. The washing step was repeated one more time. Finally, the beads were resuspended in 2 ml MES buffer, pH4.5 to 7.0.

### Example 2

### Synthesis of Polyethylene Imine-Modified Magnetic Carboxylate Beads

500 mg magnetic beads (Estapor, #39 432 084) were resuspended in 10 ml 50 mM MES buffer, pH6.1. Then, 11.5 ml of a 50 mg/ml solution of N-hydroxysulfosuccinimide (NHS) were added and the solution was mixed with a mini-shaker. Then, 10 ml of a 52 μmol/l solution of 1-ethyl-3-(3-dimethylaminopropyl)carbodiimide (EDC) were added, followed by another mixing step. The reaction mixture was incubated for 30 min on a rotating end-over end shaker. The magnetic beads were separated with a magnet and the supernatant was removed. The beads were resuspended in 50 ml 50 mM MES buffer, pH6.1 and distributed to 5 aliquots of 10 ml each. The beads were separated with a magnet and the supernatants were removed. In each aliquot the beads were resuspended in 1 ml 50 mM MES buffer, pH 6.1. Each aliquot was supplemented with 2 ml polyethylene imine (SAF, #408727) at a concentration of 500 mg/ml in 50 mM MES buffer, pH8.5. The ingredients of the aliquots were carefully mixed and treated with ultrasound for 10 min. Then the aliquots were incubated on a rotating end-over end shaker for 1 hr. The beads were separated with a magnet, and the supernatants were removed. The beads were washed with 10 ml 50 mM MES buffer, pH6.1, followed by separation with the magnet and removal of the supernatants. The washing step was repeated three more times. Finally, the beads were resuspended in 2 ml MES buffer, pH4.5 to pH7.0.

### Example 3

### Size Fractionation of DNA Fragments by Selective Binding

5 μl of a suspension comprising 0.13 mg of spermine-coated polymeric beads (synthesized according to example 1 with Seradyn SeraMag carboxy beads, amine coupling at pH 8.5) were mixed with 9 μl of a DNA-size standard (GelPilot 1 kb Plus Ladder 100 bp-10 kbp, Qiagen #239095) and 100 μl of a binding buffer (50 mM MES, pH 6.4, pH 6.6, pH 6.8 or 7.0 Samples were briefly mixed by vortexing and incubated for 1 min. Then, beads were separated with a magnet, and the supernatants were transferred to fresh tubes. Beads with bound DNA were washed with 100 μl deionized water, the beads were separated with a magnet and the supernatant was discarded. The washing step was repeated one more time, and the supernatant was discarded. The beads were resuspended in 50 μl elution buffer, briefly mixed by vortexing and incubated for 1 min. Then, the beads were separated with the magnet and the eluates were transferred to fresh tubes. For each binding condition, 6 different elution buffers were tested, namely:

Elution buffer 1: 100 mM NaCl, 50 mM TRIS pH 7.5
Elution buffer 2: 200 mM NaCl, 50 mM TRIS pH 7.5
Elution buffer 3: 100 mM NaCl, 50 mM TRIS pH 8.0
Elution buffer 4: 200 mM NaCl, 50 mM TRIS pH 8.0
Elution buffer 5: 50 mM NaCl, 50 mM TRIS pH 8.5
Elution buffer 6: 50 mM NaCl, 50 mM TRIS pH 8.5

The samples, supernatants and eluates were loaded onto an agarose gel and the DNA was separated by gel-electrophoresis. DNA fragments were visualized by ethidiumbromide staining. This allows to estimate the quantity of bound and unbound DNA from the gel photograph (see FIG. 1a and FIG. 1b, respectively). The DNA ladder profile and the corresponding DNA size is shown in FIG. 1c.

When using a binding buffer equilibrated to pH 6.4, DNA fragments shorter than 1 kb were predominantly detected in the supernatant fraction (see FIG. 1a, lanes “bind pH 6.4”), whereas DNA fragments longer than 1 kb were predominantly found in the eluates (see FIG. 1b, lanes “bind 6.4”). Therefore, at pH 6.4 nucleic acids having a size of 500 bp and more were predominantly bound to the used anion exchange particles while nucleic acids smaller than 500 to 700 bp and in particular nucleic acids smaller than 500 bp were found in the supernatant and thus were not bound to the anion exchange matrix at this size selective binding conditions. At pH 6.6, larger DNA fragments of 1600 bp and more were bound to the anion exchange matrix, while smaller fragments were found in the supernatant.

From this experiment it can be concluded that DNA of a preselected size, respectively size range can be isolated from a pool of DNA fragments with various sizes distribution by using selective binding conditions as are taught by the method according to the present invention.

### Example 4

### Size Fractionation of DNA Fragments by Selective Elution

A suspension including 0.4 mg of magnetic beads coated with different anion exchange groups were mixed with 10 μl of a DNA-size standard (such as GelPilot 1 kb Plus Ladder 100 bp-10 kbp, Qiagen #239095) and 100 μl of binding buffer (50 mM MES, pH5.8). The following bead types were tested:

Bead type I: Spermine-coated polymer particles with carboxylate surface (Seradyn SpeedBeads),
Bead type II: Spermine-coated polymer particles (Seradyn CarboxyBeads),
Bead type III: Spermine-coated polymer particles (Seradyn CarboxyBeads),
Bead type IV: Spermine-coated polymer particles (Seradyn CarboxyBeads)

The density of the spermine groups on the surface of the carboxylated beads varied between Bead type I to IV and was as follows: Bead type III>Bead type I>Bead Type IV>Bead Type II.

Samples were briefly mixed by vortexing and incubated for 1 min. Then, beads were separated with a magnet and the supernatants were transferred to a fresh tube. Beads with bound DNA were washed with 100 μl deionized water, the beads were separated with a magnet and the supernatant was discarded. The washing step was repeated one more time, and the supernatant was discarded. The beads were resuspended in 50 μl elution buffer (50 mM MES, pH6.2, pH6.3 or pH6.4) and mixed by briefly vortexing and incubated for 1 min. The beads were separated with a magnet and the eluates were transferred to fresh tubes.

For analysis, eluates were loaded onto an agarose gel and the DNA was separated by gel-electrophoresis. DNA fragments were visualized by ethidiumbromide staining. The respective quantity of purified DNA fragments can be estimated from the gel photograph (see FIGS. 2a and 2b).

The size of the eluted DNA fragments depends on the pH value of the elution buffer used. At pH6.2 only the smallest, respectively the smallest three fragments were eluted, while at pH6.3 (Bead type II) and pH6.4 (Bead type I and Bead type III) the cut-off was already at around 1 kb—the size corresponding to the thickest band of the marker. The fragments smaller than 1 kb were predominantly eluted, while the larger fragments predominantly remained bound to the magnetic beads.

In a parallel experiment it was shown, that the complete input amount of the DNA size standard was bound to the magnetic beads (see FIG. 2b). No DNA could be detected in the supernatant fractions.

### Example 5

### Size Fractionation of RNA by Selective Binding

In preparation for this experiment total RNA had been isolated from Jurkat cells with an RNA extraction kit (such as RNeasy, Qiagen).

0.25 mg of spermine-coated polymeric beads (such as Carboxyl-Adembeads, Ademtech) were mixed with 100 μl binding buffer (25 mM MES, pH5.0, pH6.0 or pH7.0) and 10 μl of an RNA mixture containing 3 μg siRNA (lamin A/C siRNA, Qiagen #1027320) and 5 μg total RNA which has been obtained from Jurkat cells using the RNeasy RNA kit (#74106). Beads from three different synthesis batches were used, designated as batches NK_20, NK_21 and NK_22. The sample ingredients were mixed by vortexing and the beads were separated with a magnet. The supernatants were removed. Beads with bound RNA were washed with 100 μl deionized water, the beads were separated with a magnet and the supernatant was discarded. The washing step was repeated one more time, and the supernatant was discarded.

The beads were resuspended in 100 μl elution buffer (50 mM MES, 100 mM NaCl, pH8.5) and mixed by vortexing. Then, the beads were separated with a magnet and the eluates were transferred to fresh tubes. For analysis, the RNA mixture, eluates and supernatants were loaded onto an agarose gel and the RNA was separated by gel-electrophoresis. RNA was visualized by ethidium bromide staining. The quantity of purified RNA can be estimated from the gel photograph (see FIGS. 3a and 3b).

From the figures one can conclude that at pH5.0 and pH6.0 only the two slower migrating RNA fragments of total RNA were bound (see FIG. 3b, lanes labelled with “bind pH 5.0” and “bind pH 6.0”) while the fast migrating short siRNA was detected only in the supernatant fractions (see FIG. 3a, lanes labelled with “flow-through bind pH 5.0” and “flow-through bind pH 6.0”). Thus, a size selective binding of RNA is possible with the method according to the present invention.

### Example 6

### Size Fractionation of DNA from Plasma

In preparation for this experiment plasma was digested with proteinase K (Qiagen) for 10 min at room temperature. 0.4 mg of magnetic beads carrying spermine anion exchange groups in 10 μl binding buffer (50 mM MES, pH6.1) were added to 20 μl digested plasma. 20 μl of a DNA size standard (GelPilot 1 kb Plus Ladder 100 bp-10 kbp, Qiagen #239095) and 80 μl binding buffer (50 mM MES, pH5.8) were added and the reagents were mixed by vortexing. The samples were incubated for 10 min with agitation at 1.000 rpm in an Eppendorf shaker. Then, beads were separated with a magnet and the supernatants were transferred to fresh tubes. The beads were washed with 100 μl deionized water, incubated for 10 min at 1.000 rpm on the shaker. Then the beads were separated with a magnet and the supernatant was discarded. The washing step was repeated one more time, and the supernatant was discarded.

The beads were resuspended in 100 μl elution buffer (50 mM MES, pH6.2, pH6.3, pH6.4, pH6.5) and incubated for 10 min with agitation at 1.000 rpm on the shaker. Then, beads were separated with a magnet and these first eluates were transferred to fresh tubes. Following the first elution process, the beads were resuspended in 100 μl of a second elution buffer (50 mM MES, 50 mM NaCl, pH8.5) and incubated for 10 min with agitation at 1.000 rpm on the shaker. Then, beads were separated with a magnet and also these second eluates were transferred to fresh tubes.

For analysis, the supernatants, the first eluates and the second eluates were loaded onto an agarose gel and the DNA was separated by gel-electrophoresis. DNA fragments were visualized by ethidiumbromide staining. The quantity of purified DNA was estimated from the gel photograph (see FIG. 4). The gel picture shows that besides minor contaminations derived from the gel pockets no DNA can be detected in the supernatants (see FIG. 4, lane labelled “flow-through bind”). Almost all DNA was bound to the beads.

Eluates of pH values in the range from pH6.3 to pH6.5 show a preferred elution of shorter fragments, wherein with increasing pH value the cut-off is shifting towards longer fragments. DNA fragments that remained bound to the beads and were not eluted with the first elution step were then detected in the eluates from the second elution step.

### Example 7

### Size Fractionation of Short DNA Fragments by Selective Binding

This experiment was performed in order to demonstrate the selective binding of DNA-fragments of a size from 50 bp to 500 bp to spermine-coated magnetic beads at pH 6.6, pH 6.7 and pH 6.8, respectively. The spermine groups were present on the bead surface at a high density.

0.25 mg of spermine-coated magnetic beads (such as Carboxyl-Adembeads, Ademtech//were mixed with 10 μl of a DNA-size standard (such as GelPilot 50 bp Ladder 50 bp-500 bp, Qiagen, see FIG. 6) and 100 μl of binding buffer (100 mM MES, pH6.6, pH6.7 and pH6.8). Samples were briefly mixed by vortexing and then incubated for 5 min at room temperature while constant shaking using a vibrating platform shaker (such as Heidolph Titramax 100, set at position 6).

After incubation, beads were separated with a magnet for 1 min. Supernatants were carefully transferred to a fresh tube. The supernatants with the unbound DNA were evaporated at 70° C. to 50 μl volume and stored for further analysis. Beads with bound DNA were washed twice with 100 μl water, each washing step including 5 min incubation on the shaker and 1 min bead separation with the magnet. The supernatant of the washing steps was discarded. Following the washing procedure, 50 μl elution buffer was added to the beads (100 mM NaCl/50 mM Tris, pH8.5), and the samples were incubated for 5 min with constant shaking. Then, beads were separated with the magnet for 1 min, and the eluates were transferred to fresh tubes.

The elution volume was determined and compared to the input volume of the elution buffer (input 50 μl). For each sample, the elution volume corresponded to 100% to the input volume (see FIG. 5). 10 μl of each eluate and supernatant were analyzed by gel-electrophoresis (see FIG. 7). As a reference, the DNA-size standard GelPilot 50 bp Ladder 50 bp-500 bp was used (see FIG. 6).

Lanes labelled 1 to 12 correspond to unbound DNA in the supernatants, while lanes labelled 13 to 24 are loaded with the eluates (see FIG. 7). Lanes 1 to 4 represent supernatant after binding at pH 6.6, lanes 5 to 8 correspond to supernatant after binding at pH 6.7 and lanes 9 to 12 correspond to supernatant after binding at pH 6.8. Accordingly, lanes 13 to 16 correspond to eluate from beads incubated with DNA at pH 6.6, lanes 17 to 20 correspond to eluate from beads incubated with DNA at pH 6.7 and lanes 21 to 24 correspond to eluate from beads incubated with DNA at pH 6.8. The amount of size standard loaded in lanes “L1” equalled to the input amount used in the binding studies (2 μl, equal to ⅕ of the ladder), while the amount loaded in lanes “L2” was 10 μl out of 50 μl total eluate volume.

When incubating DNA fragments of a size from 50 bp to 500 bp with beads in a binding buffer with a pH value of pH 6.6 or pH 6.7, the short 50 bp and 100 bp fragments were not bound to the beads and therefore are detected in the corresponding supernatant fractions (see FIG. 7, compare lanes 13 to 20 with lanes 1 to 8). The supernatants derived from both pH values also contain residual amounts of larger unbound DNA fragments, as seen by a smear-like ethidium bromide staining pattern. However, DNA fragments of 150 bp and larger were bound to some extent by the beads at both pH 6.6 and pH 6.7, while strong binding was observed for fragments of size 300 bp and larger.

Of DNA fragments incubated with beads at pH6.8 fragments of size 300 bp and larger were bound to the beads (see FIG. 7, compare lanes 21 to 24 with L1). Furthermore, DNA binding to beads at pH6.8 was less strong.

In summary, short DNA fragments of 50 bp and 100 bp can selectively be depleted from a mixture of fragments by binding to spermine-coated beads. Only DNA fragments of 150 bp and larger were bound and eluted, while smaller fragments do not bind and remain in the unbound fraction.

### Example 8

### Size Fractionation of Short DNA Fragments by Selective Binding and Elution

This experiment was based on the observation that using appropriate binding conditions DNA fragments of 50 bp and 100 bp can selectively be depleted from a mixture of DNA fragments by selective binding to spermine-coated beads in an appropriate binding buffer with a pH6.6 or pH6.7 (see example 7). Fragments were eluted with an elution buffer composed of 100 mM NaCl/50 mM Tris, pH8.5. Here, a similar experiment is performed with the same binding conditions but a modified elution step, in which the bound fragments are eluted with 20 mM KCl/50 mM Tris either at pH8.5 or at pH9.0.

0.25 mg of spermine-coated magnetic beads (Seradyn Beads, Thermo, Fremont, Calif.) were mixed with 10 μl of a DNA-size standard (for example GelPilot 50 bp Ladder 50 bp-500 bp, Qiagen, see FIG. 6) and 100 μl of binding buffer (100 mM MES, pH6.6 or pH6.7). Samples were briefly mixed by vortexing and then incubated for 5 min at room temperature while constant shaking using a vibrating platform shaker (such as Heidolph Titramax 100, set at position 6).

After incubation, beads were separated with a magnet for 1 min. Supernatants were carefully transferred to a fresh tube. The supernatants with the unbound DNA were evaporated at 70° C. to 50 μl volume and stored for further analysis. Beads with bound DNA were washed twice with 100 μl water, each washing step including 5 min incubation on the shaker and 1 min bead separation with the magnet. The supernatant of the washing steps was discarded. Following the washing procedure, 50 μl elution buffer was added to the beads (either 20 mM KCl/50 mM Tris, pH8.5 or 20 mM KCl/50 mM Tris, pH9.0), and the samples were incubated for 5 min with constant shaking. Then, the beads were separated with the magnet for 1 min, and the eluates were transferred to fresh tubes.

The elution volume was determined and compared to the input volume of the elution buffer (input 50 μl). For each sample, the elution volume corresponded to the input volume (see FIG. 8).

10 μl of each eluate and supernatant were analyzed by gel-electrophoresis (see FIG. 9). Lanes labelled 1 to 12 correspond to unbound DNA in the supernatants, while lanes 13 to 24 were loaded with eluates. Lanes 1 to 3 represent supernatant after binding at pH6.6 and elution at pH8.5, lanes 4 to 6 correspond to supernatant after binding at pH6.6 and elution at pH9.0, lanes 7 to 9 correspond to supernatant after binding at pH6.7 and elution at pH8.5, and lanes 10 to 12 correspond to supernatant after binding at pH6.7 and elution at pH9.0. Accordingly, lanes 13 to 15 correspond to eluate from beads incubated with DNA at pH6.6 and eluted at pH8.5, lanes 16 to 18 correspond to eluate from beads incubated with DNA at pH6.6 and eluted at pH9.0, lanes 19 to 21 correspond to eluate from beads incubated with DNA at pH6.7 and eluted at pH8.5, and lanes 22 to 24 correspond to eluate from beads incubated with DNA at pH6.7 and eluted at pH9.0.

As a reference, a DNA-size standard was used (such as GelPilot 50 bp Ladder, 50 bp-500 bp, Qiagen/see FIG. 6). The amount of size standard loaded in lanes “L1” was equal to the input amount used in the binding studies (2 μl, equals ⅕ of size standard used in the procedure), while the amount loaded in lanes “L2” was 10 μl out of 50 μl total eluate volume (see FIG. 9).

Again it was demonstrated, that DNA fragments of 50 bp and 100 bp can be entirely depleted from a mixture of fragments using binding conditions of 100 mM MES pH6.6 or pH6.7. In addition, fragments of 150 bp can be partially depleted (FIG. 9, lanes 13 to 24). The depleted fragments were detected in the supernatants (FIG. 9, lanes 1 to 12). The supernatants also contain residual amounts of other DNA fragments, as seen by a smear-like ethidiumbromide staining pattern. Comparing the DNA patterns, no major differences in DNA fragment depletion between samples eluted with pH8.5 and pH9.0 are observed.

### BRIEF DESCRIPTION OF THE DRAWINGS

FIG. 1a and FIG. 1b are photographs of stained agarose gels loaded with DNA samples from a size fractionation experiment (see example 3). FIG. 1c shows the used ladder.

FIG. 2a and FIG. 2b are pictures of stained agarose gels loaded with samples of two DNA fractionation experiments that were performed in parallel (see example 4). Therein DNA fragments were bound to beads and then selectively eluted with buffer of a pH ranging from pH6.2 to pH6.4.

FIG. 3a and FIG. 3b depict stained agarose gels with samples from an RNA size fractionation experiment (see example 5).

FIG. 4 shows a stained agarose gel with samples derived from a DNA size fractionation experiment from plasma (see example 6).

FIG. 5 shows the elution results from DNA samples incubated with spermine-coated beads at pH6.6, pH6.7 and pH6.8 (see example 7).

FIG. 6 is a gel picture of the DNA-size standard used in example 7 and example 8. Each μl of the DNA standard contains 16.67 ng for the 300 bp fragment and 8.3 ng for any other fragment.

FIG. 7 is a photograph of an agarose gel with the comparative analysis of bound and unbound DNA fragments derived from a binding assay with spermine-coated magnetic beads in binding buffer at pH6.6, pH6.7 and pH6.8 (see example 7).

FIG. 8 shows the elution results from DNA samples incubated with spermine-coated beads at pH6.6, pH6.7 and pH6.8 and eluted either with 20 mM KCl/50 mM Tris, pH8.5 or with 20 mM KCl/50 mM Tris, pH9.0 (see example 8).

FIG. 9 is an agarose gel picture with a comparative analysis of bound and unbound DNA fragments after incubation with spermine-coated magnetic beads and elution with different elution buffers (see example 8).

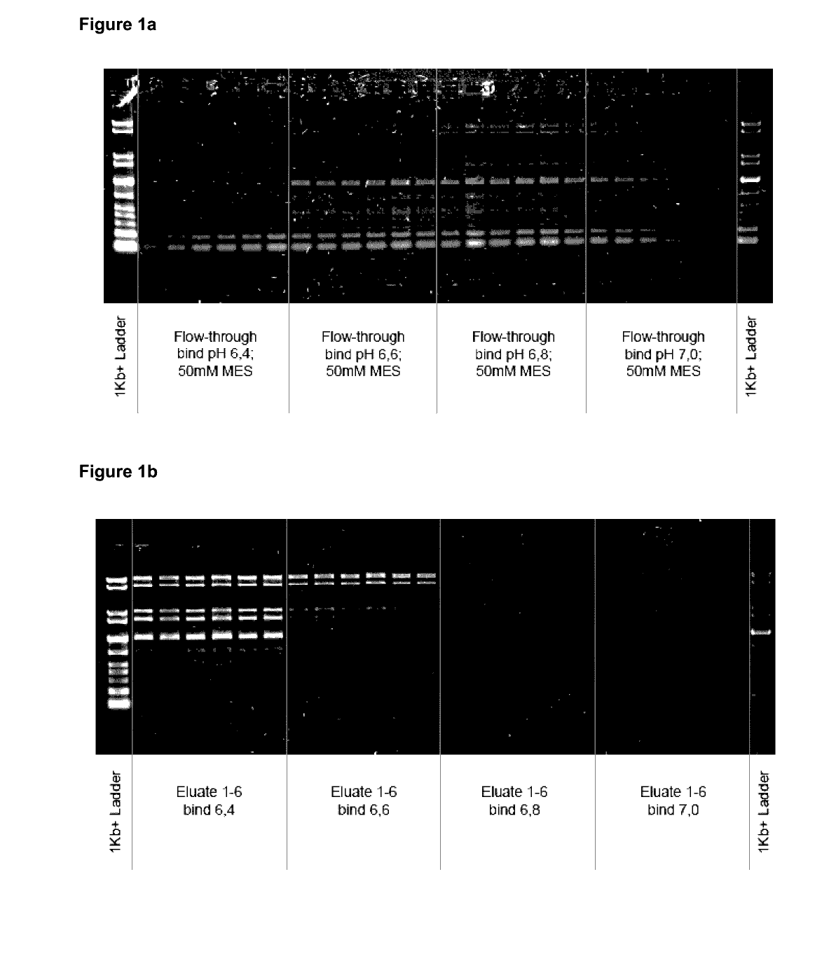

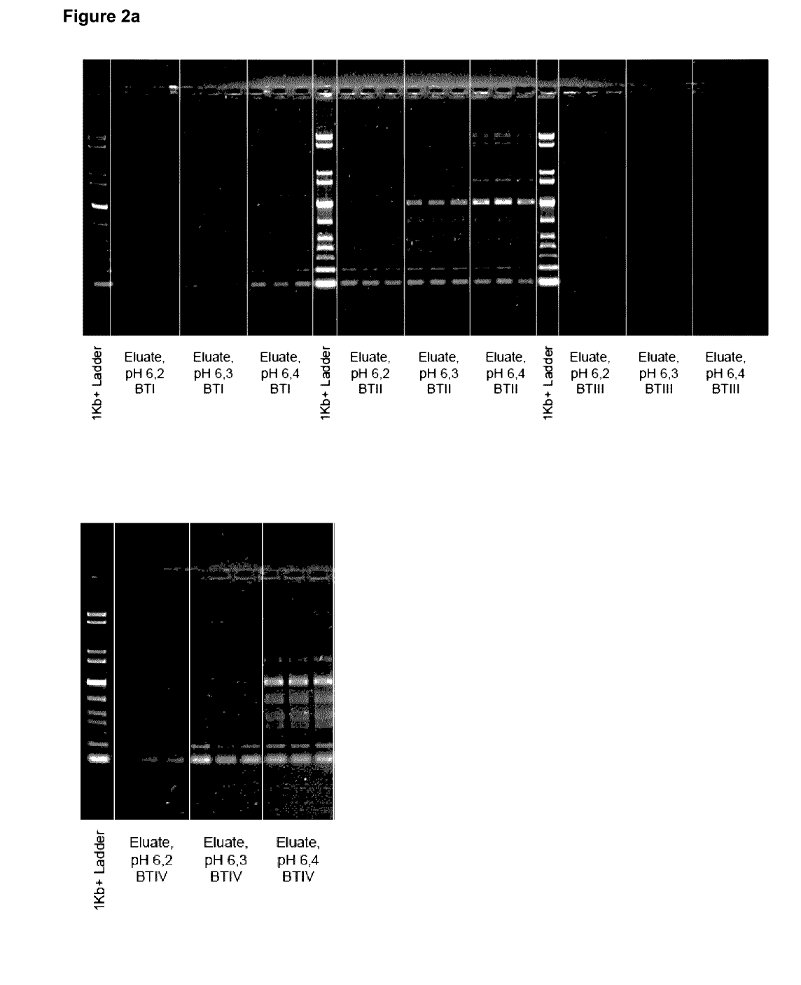

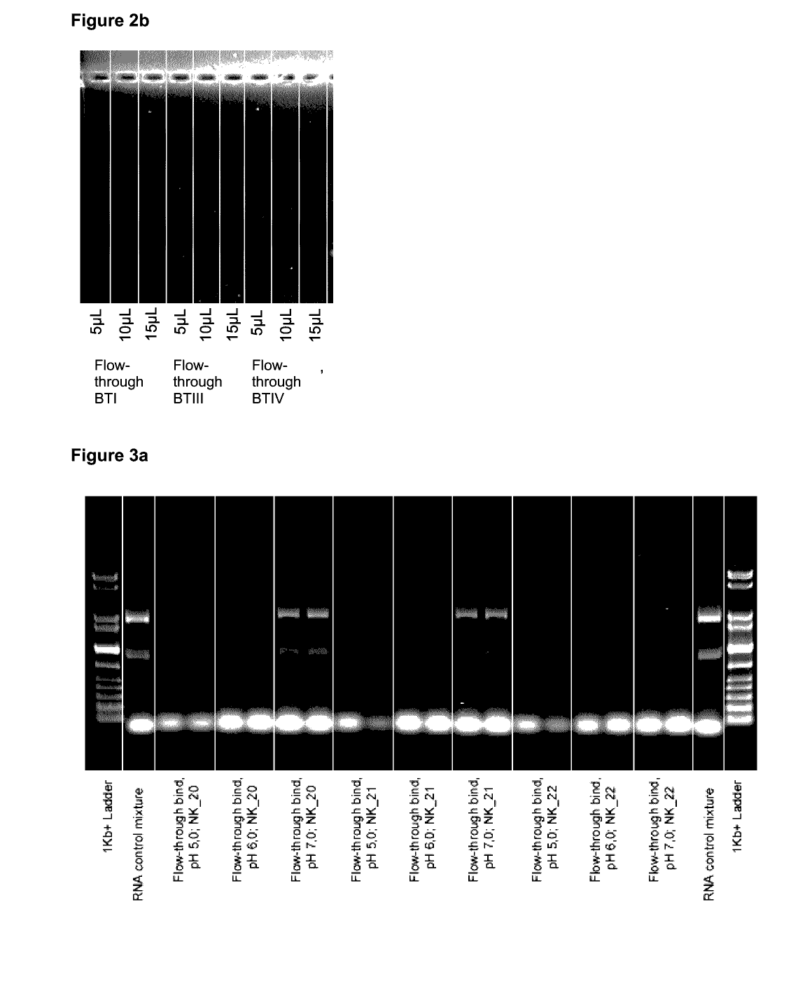

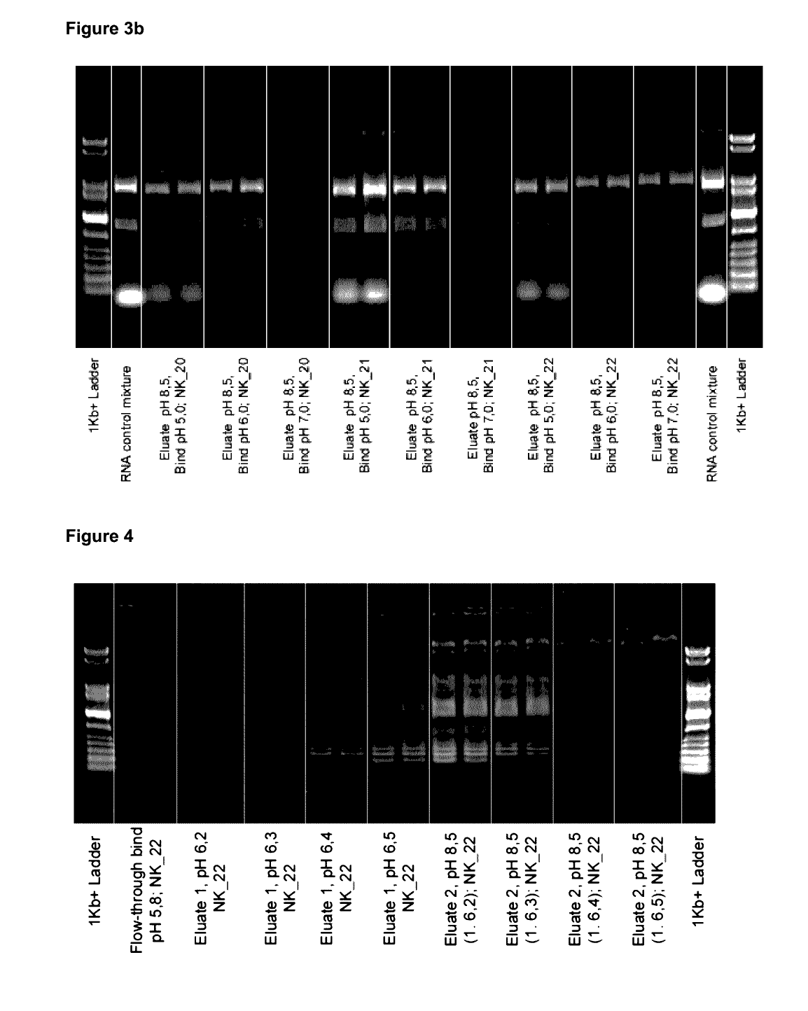

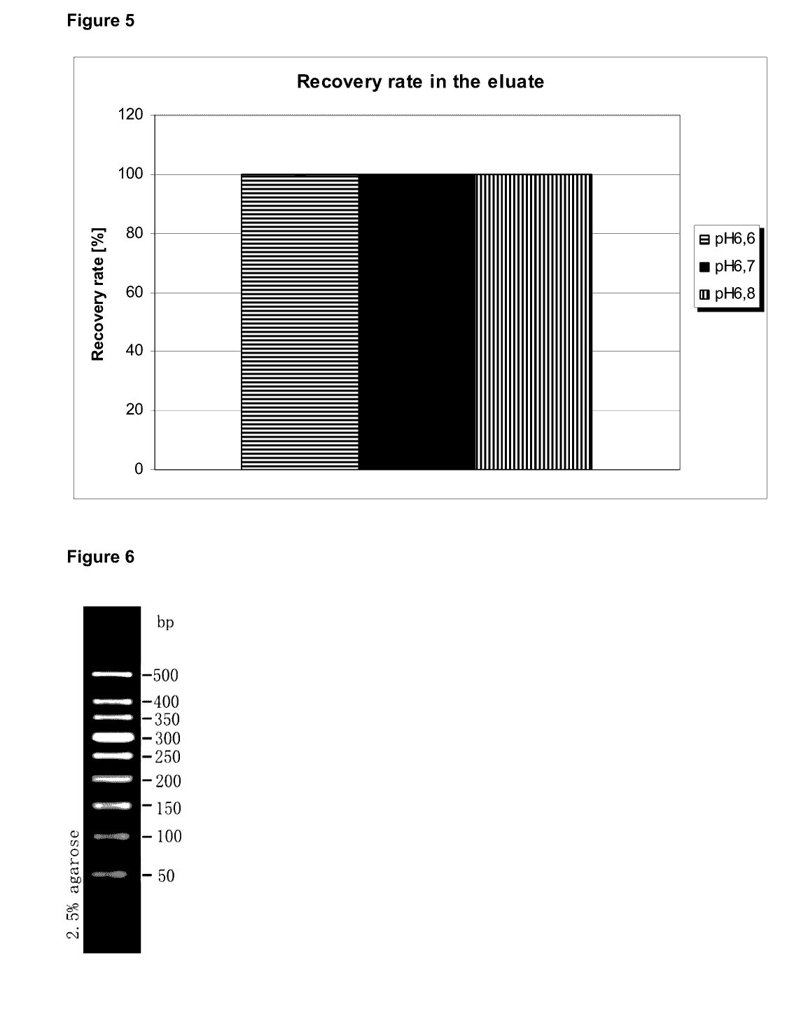

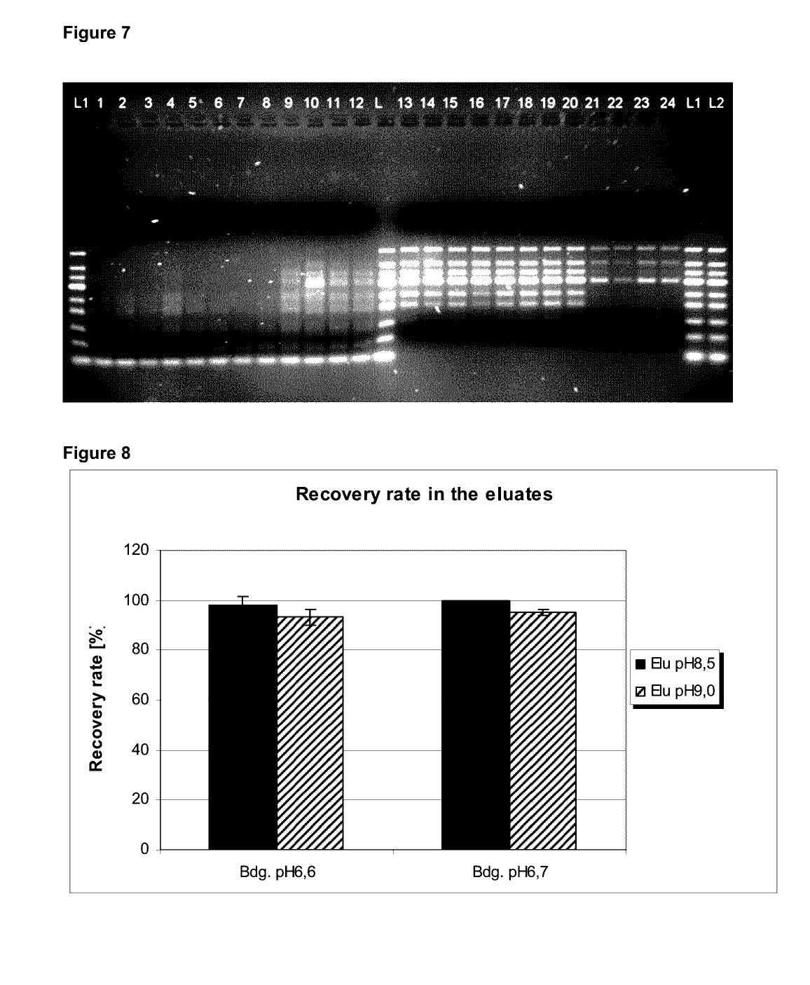

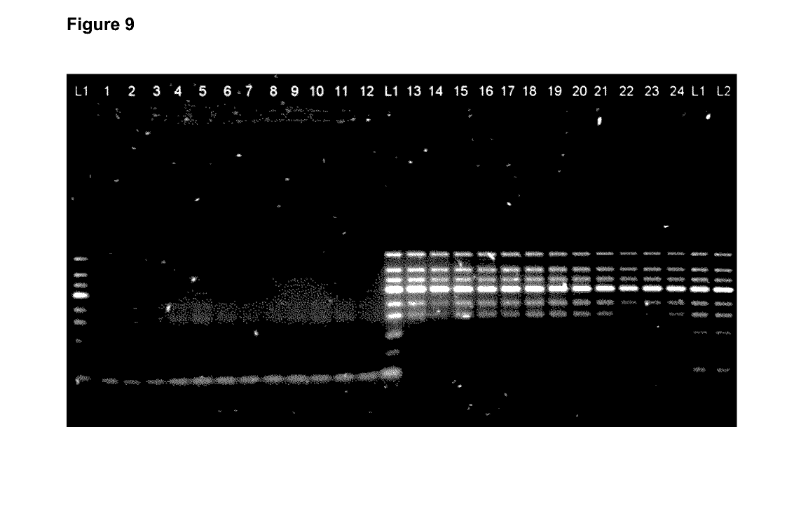

1.-17. (canceled)

18. A method for isolating nucleic acids by size from a sample comprising nucleic acids of different sizes using an anion exchange matrix, wherein the anion exchange matrix is a solid phase comprising anion exchange groups and wherein magnetic particles are used as solid phase, wherein nucleic acids of a preselected size or a preselected size range are isolated by adjusting the pH value during elution and/or binding, comprising:
(A) a) binding nucleic acids of different sizes to an anion exchange matrix at a first pH wherein nucleic acids bind to the anion exchange matrix, and
b) selectively eluting nucleic acids of a preselected size or a preselected size range from the anion exchange matrix using a second pH which is higher than the first pH, wherein the average length of the nucleic acids eluted from the anion exchange matrix is shorter than the average length of the nucleic acids which remain bound to the solid phase; or
(B) a) selectively binding nucleic acids of a preselected size or a preselected size range to an anion exchange matrix at a first pH, wherein the average length of the nucleic acids that bind to the anion exchange matrix under the chosen binding conditions is longer than the average length of nucleic acids which are not bound to the anion exchange matrix, and
b) separating the bound nucleic acids from the remaining sample.

19. The method according to claim 18, wherein the method comprises steps (A) a) and (A) b), and wherein the second pH is used during elution so that predominantly longer nucleic acid molecules having a length above a defined cut-off value remain bound to the anion exchange matrix during elution while smaller nucleic acids having a length below said cut-off value are predominantly eluted.

20. The method according to claim 18, wherein the method comprises steps (A) a) and (A) b), and has one or more of the following characteristics:
a) in step A) a), the first pH at which binding is performed is below 7, below 6.5 or below 6;
b) the anion exchange matrix with the bound nucleic acids is separated from the remaining sample, and the bound nucleic acids are optionally washed;
c) the length of the nucleic acids that are predominantly eluted in step (A) b) is controlled by the choice of the second pH value;
d) the second pH value that is used in elution step (A) b) is at least 0.2 pH units, at least 0.3 pH units, at least 0.4 pH units or at least 0.5 pH units higher than the first pH value;
e) elution step (A) b) is performed with an elution buffer having a constant pH value;
f) the method further comprises elution step (A) c) wherein at least a portion of the nucleic acids that remain bound to the anion exchange matrix after elution step (A) b) is eluted; and/or
g) nucleic acids that remain bound to the anion exchange matrix after elution step (A) b) are discarded.

21. The method according to claim 20, wherein in characteristic d), the second pH value that is used in elution step (A) b) is not more than 3 pH units, not more than 2.5 pH units, not more than 2 pH units, not more than 1.5 pH units, not more than 1 pH unit or not more than 0.75 pH units higher than the first pH value.

22. The method according to claim 18, wherein the method comprises steps (B) a) and (B) b), and the first pH is used during binding so that predominantly longer nucleic acid molecules having a length above a defined cut-off value bind to the anion exchange matrix while smaller nucleic acids having a length below said defined cut-off value are predominantly not bound.

23. The method according to claim 18, wherein the method comprises steps (B) a) and (B) b), and has one or more of the following characteristics:
a) the length of the nucleic acids that are predominantly bound in step (B) a) is controlled by the choice of the first pH value;
b) the first pH value that is used in binding step (B) a) lies in a pH range from 5 to 8;
c) the anion exchange matrix with the bound nucleic acids is separated from the remaining sample, and the bound nucleic acids are optionally washed;
d) nucleic acids remaining in the supernatant are isolated therefrom; and/or
e) the anion exchange matrix with the bound nucleic acids is discarded.

24. The method according to claim 23, wherein in characteristic b), the first pH value that is used in binding step (B) a) lies in a pH range from 5 to 7.

25. The method according to claim 23, wherein in characteristic b), the first pH value that is used in binding step (B) a) lies in a pH range from 6 to 6.7.

26. The method according to claim 18, wherein the method comprises steps (B) a) and (B) b), and further comprises elution step (B) c) wherein at least a portion of the nucleic acids that were bound to the anion exchange matrix in binding step (B) a) is eluted.

27. The method according to claim 26, further comprising a size selective elution step having one or more of the following characteristics:
a) the size selective elution step is performed using a second pH which is higher than the first pH, wherein the average length of the nucleic acids eluted from the anion exchange matrix is shorter than the average length of the nucleic acids which remain bound to the solid phase;
b) the size selective elution step is performed using a second pH so that predominantly longer nucleic acid molecules having a length above a defined cut-off value remain bound to the anion exchange matrix during elution while smaller nucleic acids having a length below said cut-off value are predominantly eluted;
c) the size selection elution step is performed using a second pH, wherein the length of the nucleic acids that are predominantly eluted is controlled by the choice of the second pH value, and wherein the second pH value is higher than the first pH value;
d) the size selection elution is performed using a second pH, wherein the second pH value is at least 0.2 pH units, at least 0.3 pH units, at least 0.4 pH units or at least 0.5 pH units higher than the first pH value;
e) the size selection elution is performed with an elution buffer having a constant pH value;
f) at least a portion of the nucleic acids that remain bound to the anion exchange matrix after elution step (B) b) is eluted in the size selection elution; and/or
g) the nucleic acids that remain bound to the anion exchange matrix after the size selection elution are discarded.

28. The method according to claim 27, wherein in characteristic d), the second pH value is not more than 3 pH units, not more than 2.5 pH units, not more than 2 pH units, not more than 1.5 pH units, not more than 1 pH unit or not more than 0.75 pH units higher than the first pH value.

29. The method according to claim 18, wherein the sample is lysed prior to binding step (A) a) or (B) a).

30. The method according to claim 18, wherein the binding of step (A) a) or step (B) a) is performed in the absence of alcohol and/or chaotropic salts.

31. The method according to claim 18, wherein the first pH at which the nucleic acids are bound to the anion exchange groups is below the pKa value of a protonatable group of the anion exchange groups.

32. The method of claim 31, wherein the second pH at which elution of the nucleic acids is achieved is higher than the first pH but below the pKa value of a protonatable group of the anion exchange groups.

33. The method according to claim 18, wherein the anion exchange matrix is a solid phase that comprises anion exchange groups and has one or more of the following characteristics:
a) the solid phase carries a type of anionic exchange group that is positively charged at the first pH;
b) the solid phase carries a type of anionic exchange group which comprises a protonatable group;
c) the anion exchange groups comprise at least one primary, secondary or tertiary amine group;
d) the anion exchange groups are selected from the group consisting of aminomethyl (AM), aminoethyl (AE), aminoalkyl, alkylaminoalkyl, dialkylaminoalkyl, diethylaminoethyl (DEAE), ethylendiamine, diethylentriamine, triethylentetraamine, tetraethylenpentaamine, pentaethylenhexaamine, trimethylamino (TMA), triethylaminoethyl (TEAE), linear or branched polyethylenimine (PEI), carboxylated or hydroxyalkylated polyethylenimine, jeffamine, Tris, Bis-Tris, spermine, spermidine, 3-(propylamino)propylamine, polyamidoamine (PAMAM) dendrimers, polyallylamine, polyvinylamine, N-morpholinoethyl, polylysine, and tetraazacycloalkanes, cyclic amines and protonatable aromatic amines;
e) the solid phase comprises a carboxylated surface comprising amine groups as anion exchange groups;
f) the solid phase comprises spermine groups as anion exchange groups and preferably comprises carboxyl groups on its surface;
g) the solid phase comprises a silica surface comprising aminoalkylsilane groups as anion exchange groups and dihydroxypropyloxy-propylsilanes; and/or
h) the solid phase additionally carries inert ligands to reduce the amount of nucleic acid binding anion exchange groups and/or functional groups which assist the elution of the bound nucleic acids at the second pH value.

34. The method of claim 33, wherein in characteristic b), the pKa value of the protonatable group is in the range of from 8 to 12.

35. The method of claim 33, wherein in characteristic b), the pKa value of the protonatable group is in the range of from 9 to 11.

36. The method of claim 33, wherein in characteristic e), the anion exchange group comprises 2 to 6 amino groups.

37. The method of claim 33, wherein in characteristic h), the functional groups are carboxyl groups.

38. The method according to claim 18, wherein the anion exchange matrix is provided by magnetic particles comprising a coating with silica, polysilicic acid, glass or polymeric material to which the anion exchange groups are covalently attached, optionally via a linker group; and wherein the anion exchange groups comprise 2 to 6 primary and/or secondary amino groups.

39. The method according to claim 38, wherein the anion exchange groups are spermine or spermidine.

40. The method according to claim 18, wherein the isolated nucleic acid molecules of a preselected size or preselected size range are used in a sequencing reaction.

41. The method according to claim 40, wherein the sequencing reaction is a next generation sequencing reaction.
